(19) United States
(12) Patent Application Publication
(10) Pub. No.: US 2008/0294150 A1
(43) Pub. Date: Nov. 27, 2008

(54) Photoselective Islets In Skin And Other Tissues

(75) Inventors: Gregory B. Altshuler, Lincoln, MA (US); Ilya Yaroslavsky, North Andover, MA (US); James Childs, Bolton, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: PALOMAR MEDICAL TECHNOLOGIES, INC., Burlington, MA (US)

(21) Appl. No.: 12/102,636

(22) Filed: Apr. 14, 2008

Related U.S. Application Data
(63) Continuation-in-part of application No. 11/966,538, filed on Dec. 28, 2007.
Application No. 11/966,538 is a continuation-in-part of application No. 11/966,625, filed on Dec. 28, 2007.
Application No. 11/966,625 is a continuation-in-part of application No. 11/097,841, filed on Apr. 1, 2005.
Application No. 11/097,841 is a continuation-in-part of application No. 11/098,000, filed on Apr. 1, 2005.
Application No. 11/098,000 is a continuation-in-part of application No. 11/098,036, filed on Apr. 1, 2005.
Application No. 11/098,036 is a continuation-in-part of application No. 11/098,015, filed on Apr. 1, 2005.
Application No. 11/966,538 is a continuation-in-part of application No. 11/097,841, filed on Apr. 1, 2005.
Application No. 11/097,841 is a continuation-in-part of application No. 11/098,000, filed on Apr. 1, 2005.
Application No. 11/098,000 is a continuation-in-part of application No. 11/098,036, filed on Apr. 1, 2005.
Application No. 11/098,036 is a continuation-in-part of application No. 11/098,015, filed on Apr. 1, 2005.
Application No. 11/966,625 is a continuation-in-part of application No. 11/097,841, filed on Apr. 1, 2005.
Application No. 11/097,841 is a continuation-in-part of application No. 11/098,000, filed on Apr. 1, 2005.
Application No. 11/098,000 is a continuation-in-part of application No. 11/098,036, filed on Apr. 1, 2005.
Application No. 11/098,036 is a continuation-in-part of application No. 11/098,015, filed on Apr. 1, 2005.
(60) Provisional application No. 60/923,093, filed on Apr. 12, 2007.
(60) Provisional application No. 60/877,826, filed on Dec. 29, 2006.
(60) Provisional application No. 60/877,826, filed on Dec. 29, 2006.

(51) Int. Cl. A61B 18/20; A61B 18/18

(52) U.S. Cl. 606/3; 606/9

(57) ABSTRACT

Methods of treatment of tissue with electromagnetic radiation (“EMR”) to produce lattices of photoselective islets and other energy selective islets in tissue are disclosed. Also disclosed are devices and systems for producing lattices of EMR-treated islets in tissue, and cosmetic and medical applications of such devices and systems.

### RELATED APPLICATIONS

This application claims the benefit of U.S. Provisional Application No. 60/923,093, filed Apr. 12, 2007.

This application is a continuation-in-part application of U.S. application Ser. Nos. 11/966,538 and 11/966,625 (the '538 and '625 applications) that were each filed on Dec. 28, 2007 and entitled “Methods and Devices for Fractional Ablation of Tissue”, each of which claim priority to U.S. Provision Application No. 60/877,826 filed Dec. 28, 2006 and entitled “Methods And Products For Ablating Tissue Using Lattices Of EMR-Treated Islets.”

This application as well as the '538 and '625 applications are continuation-in-part applications of U.S. application Ser. Nos. 11/097,841, 11/098,000, 11/098,036, and 11/098,015, each of which was filed Apr. 1, 2005 and entitled “Methods and products for producing lattices of EMR-treated islets in tissues, and uses therefore” and each of which claims priority to U.S. Provisional Application No. 60/561,052, filed Apr. 9, 2004, U.S. Provisional Application No. 60/614,382, filed Sep. 29, 2004, U.S. Provisional Application No. 60/641,616, filed Jan. 5, 2005, and U.S. Provisional Application No. 60/620,734, filed Oct. 21, 2004.

Each of the applications and provisional applications identified above is incorporated herein by reference in its entirety.

### BACKGROUND OF THE INVENTION

1. Field of the Invention

The invention relates to the treatment of tissue with electromagnetic radiation (“EMR”) to produce lattices of EMR-treated islets in the tissue. The invention also relates to devices and systems for producing lattices of EMR-treated islets in tissue, and cosmetic and medical applications of such devices and systems.

2. Description of the Related Art

Electromagnetic radiation, particularly in the form of laser light, has been used in a variety of cosmetic and medical applications, including uses in dermatology, dentistry, opthalmology, gynecology, otorhinolaryngology and internal medicine. For most dermatological applications, the EMR treatment can be performed with a device that delivers the EMR to the surface of the targeted tissues. For applications in internal medicine, the EMR treatment is typically performed with a device that works in combination with an endoscope or catheter to deliver the EMR to internal surfaces and tissues. As a general matter, the EMR treatment is typically designed to (a) deliver one or more particular wavelengths (or a particular continuous range of wavelengths) of EMR to a tissue to induce a particular chemical reaction, (b) deliver EMR energy to a tissue to cause an increase in temperature, or (c) deliver EMR energy to a tissue to damage or destroy cellular or extracellular structures.

Until recently, all photothermal applications of light in medicine have been based on one of three approaches. The first approach, known as the principle of selective photothermolysis, sets specific requirements for the wavelengths used (which need to be absorbed preferentially by chromophores in the target area) and for the duration of the optical pulse (which needs to be shorter than characteristic thermal relaxation time of the target area). This approach was later extended, and is often called the extended theory of selective photothermolysis, to encompass situations in which the target area and target chromophore are physically separated. The second approach relies on heat diffusion from the target chromophore to the target area. The third approach relies on absorption by a chromophore which is substantially uniformly present in the tissue (e.g., water). In this last case, the damage zone can, in principle, be controlled by manipulating wavelength, fluence, incident beam size, pulse width, and cooling parameters. All three approaches have drawbacks, the most significant of which is the difficulty in eliminating unwanted side effects.

These three approaches have been used for many dermatological applications, such as hair removal, acne treatment, wrinkle treatment, and skin rejuvenation. Absorption of optical energy is also used for medical procedures, such as in the treatment of prostate cancer and certain gynecological procedures. For example, photoselective vaporization of tissue, such as prostate tissue, is based upon applying a high intensity radiation to prostate tissue using a radiation that is highly absorptive in the tissue, while being absorbed to a negligible degree by water or other irrigant during the operation, at power densities such that the majority of the energy is converted to vaporization of the tissue without significant residual coagulation of adjacent tissue.

In procedures that employ selective photothermolysis, the wavelength selected for the radiation is generally dictated by the absorption characteristics of the chromophore and may not be optimal for other purposes. Skin is a scattering medium, but such scattering is far more pronounced at some wavelengths than at others. Wavelengths preferentially absorbed by melanin, for example, are also wavelengths at which substantial scattering occurs. This is also true for the wavelengths typically utilized for treating vascular lesions. Photon absorption in skin also varies over the visible wavelength band, and some wavelengths typically used in selective photothermolysis are wavelengths at which skin is highly absorbent. The fact that wavelengths typically utilized for selective photothermolysis are highly scattered and/or highly absorbed limits the ability to selectively target body components and, in particular, limits the depths at which treatments can be effectively and efficiently performed.

Further, much of the energy applied to a target region is either scattered and does not reach the body component undergoing treatment, or is absorbed in overlying or surrounding tissue. This means that larger and more powerful EMR sources are required in order to achieve a desired therapeutic result. However, increasing power generally causes undesired and potentially dangerous heating of tissue. Thus, increasing efficacy often decreases safety, and additional cost and energy are utilized to mitigate the effects of this undesired tissue heating by surface cooling or other suitable techniques. Heat management for the more powerful EMR source is also a problem, generally requiring expensive and bulky water circulation or other heat management mechanisms. A technique which permits efficacious power levels and minimizes undesired heating, such as the bulk heating of tissue caused by water absorption, is therefore desirable.

Absorption of optical energy by water is widely used in two approaches for skin rejuvenation: ablative skin resurfacing, typically performed with either CO₂ (10.6μ) or Er:YAG (2.94μ) lasers, and non-ablative skin remodeling using a combination of deep skin heating with light from Nd:YAG (1.34μ), Er:glass (1.56μ) or diode laser (1.44μ) and skin surface cooling for selective damage of sub-epidermal tissue. Usually, primary absorption of optical energy by water causes bulk tissue damage.

The principal difference between the two techniques is the region of body where damage is initiated. In the resurfacing approach, the full thickness of the epidermis and a portion of upper dermis are ablated and/or coagulated. For cosmetic and dermatological treatments, the most effective treatments have involved the use of pulsed dye lasers. However, when using such lasers, it can be difficult to protect the epidermis. Further, when the upper layers of skin tissue coagulate, the coagulated tissue forms a barrier making it more difficult to reach deeper layers with EMR.

In the non-ablative approach, the zone of damage is shifted deeper into the tissue, with the entire epidermis being left intact. In practice, this is achieved by using different wavelengths: very shallow-penetrating ones in the ablative techniques (absorption coefficients of ˜900 cm⁻¹ and ˜13000 cm⁻¹ for CO₂ and Er:YAG wavelengths, respectively) and deeper-penetrating ones in the non-ablative modalities (absorption coefficients between 5 and 25 cm⁻¹). In addition, contact or spray cooling is applied to skin surface in non-ablative techniques, providing thermal protection for the epidermis.

Non-ablative dermal treatments are complicated by the fact that chromophore concentrations in a target (e.g., melanin in hair follicles) vary significantly from target to target and from patient to patient, making it difficult to determine optimal, or even proper, parameters for effective treatment of a given target. High absorption by certain types of skin, for example dark skinned individuals or people with very tan skin, often makes certain treatments difficult to safely perform.

### SUMMARY OF THE INVENTION

The present invention derives, in part, from the discovery that, when using electromagnetic radiation (“EMR”) to treat tissues, there are substantial advantages to producing lattices of EMR-treated islets in the tissue rather than large, continuous regions of EMR-treated tissue. The lattices are periodic patterns of islets in one, two or three dimensions in which the islets correspond to local maxima of EMR-treatment of tissue. The islets are separated from each other by non-treated tissue (or differently- or less-treated tissue). The EMR-treatment results in a lattice of EMR-treated islets which have been exposed to a particular wavelength or spectrum of EMR, and which is referred to herein as a lattice of “islets.” By producing EMR-treated islets rather than continuous regions of EMR-treatment, more EMR energy can be delivered to an islet without producing a thermal islet or damage islet, and/or the risk of bulk tissue damage can be lowered. Thus, various embodiments, examples of which are described in greater detail below, include improved devices and systems for producing lattices of EMR-treated islets in tissues, and improved cosmetic and medical applications of such devices and systems.

One embodiment is a method for treating a subvolume of tissue located below a surface of the tissue comprising irradiating the tissue with optical radiation that is more readily absorbed by the subvolume of tissue than by portions of the tissue surrounding the subvolume. The optical radiation creates a plurality of treatment zones within the subvolume of tissue separated by substantially untreated tissue within the subvolume, and the portions of tissue surrounding the subvolume of tissue are substantially untreated.

Preferred embodiments of this embodiment can include one or more of the following. The treatment zones can be regularly spaced or irregularly spaced. The treatment zones can have a width of between approximately 1 and 1000 micrometers, or more preferable between approximately 30 and 100 micrometers. The treatment zones can be located within the dermis, the epidermis, subcutaneous tissue or a combination of skin tissues. The subvolume of tissue being treated can be a lesion, including, without limitation, a vascular or pigmented lesion. Other structures such as a vein can also be treated. The treatment zones can have a fill factor in a cross-sectional plane extending through the treatment zones of between approximately 1 percent and 90 percent, or more preferably of between approximately 1 percent and 50 percent. The optical radiation can have a wavelength of 1064 nanometers, and can be either coherent (as from a laser) or incoherent (as from a flashlamp). The treatment zone can include thermally injured tissue, coagulated tissue, denatured tissue, or ablated tissue. The substantially untreated portions of tissue can contain zones of thermally heated tissue resulting from the step of irradiation or other forms of thermal effect or damage.

Another embodiment is a method for treating a subsurface tissue comprising irradiating a surface of a tissue with electromagnetic radiation that is transmitted to a subsurface tissue via an intervening tissue located between the surface and the subsurface tissue, where the electromagnetic radiation is more preferentially selected by the subsurface tissue than by the intervening tissue. The electromagnetic radiation creates a plurality of damage zones within the subsurface tissue separated by undamaged tissue within the subvolume, and the intervening tissue is undamaged.

Preferred embodiments of this embodiment can include one or more of the following. The damage zones can be regularly spaced from each other. The damage zones can have a width of between approximately 1 and 1000 micrometers, or more preferable between approximately 30 and 100 micrometers. The damage zones can be located within the dermis, the epidermis, subcutaneous tissue or a combination of skin tissues. The subsurface tissue can be a lesion, including, without limitation, a vascular or pigmented lesion. Other structures such as a vein can also be treated. The damage zones can have a fill factor in a cross-sectional plane extending through the damage zones of between approximately 1 percent and 90 percent, or more preferably of between approximately 1 percent and 50 percent. The electromagnetic radiation can have a wavelength of 1064 nanometers, and can be either coherent (as from a laser) or incoherent (as from a flashlamp). The damage zone can include thermally injured tissue, coagulated tissue, denatured tissue, or ablated tissue. The intervening tissue can contain zones of thermally heated tissue resulting from the step of irradiation or other forms of thermal effect. The tissue can be irradiated by scanning the electromagnetic radiation to an array of locations on the surface of the tissue corresponding to the damage zones within the subsurface tissue, or, alternatively, the tissue can be irradiated with an array of beams of electromagnetic radiation that create the damaged zones within the subsurface tissue.

Another embodiment is a method for treating a subvolume of tissue located below a surface of the tissue comprising irradiating the tissue with electromagnetic radiation, and creating an array of treatment zones within the subvolume of tissue separated by other tissue of the subvolume. A portion of tissue surrounding the subvolume includes zones of differently-treated tissue.

Preferred embodiments of this embodiment can include one or more of the following. The other tissue of the subvolume can be untreated, heated, or differently-treated. The portion of tissue surrounding the subvolume can also be untreated, heated, or differently treated. The portions of tissue surrounding the subvolume can contain zones of heated tissue corresponding to the treatment zones of the subvolume, and can contain zones of damaged tissue (such as coagulated or denatured tissue) corresponding to the treatment zones of the subvolume, wherein the degree of damage in zone of damaged tissue in the portions of tissue surrounding the subvolume is less than the degree of damage in the treatment zones of the subvolume.

Another embodiment is a device for treating soft tissue that can have a source of electromagnetic radiation, an output aperture, and a transmission path extending from the source of the electromagnetic radiation to the output aperture that is configured to deliver the electromagnetic radiation to the soft tissue. The output aperture is configured to emit electromagnetic radiation in a pattern of spots on a tissue surface, and the source is configured to generate electromagnetic radiation that is selectively absorbed by a subvolume of tissue located below a surface of the soft tissue.

Preferred embodiments of this embodiment can include one or more of the following. The source can be configured to produce coherent radiation (such as by a laser) or in coherent radiation (such as by a flashlamp). The electromagnetic radiation can have a wavelength of between approximately 190 nanometers and 100 micrometers, and, more preferably, have a wavelength in the infrared range, and, even more preferably, have a wavelength of approximately 1064 nanometers. The transmission path can include an array of lenses to generate beams of electromagnetic radiation corresponding to the pattern of spots on the tissue surface, and the transmission path can include a scanning device to generate an array of beams of electromagnetic radiation corresponding to the pattern of spots on the tissue surface.

### BRIEF DESCRIPTION OF THE DRAWINGS

The following drawings are illustrative of embodiments of the invention and are not meant to limit the scope of the invention as encompassed by the claims.

FIG. 1 is a diagram showing an exemplary cross-section of human skin.

FIG. 2 is a schematic diagram showing the layers of skin.

FIG. 3 is a schematic view of various embodiments of micro-islets.

FIG. 4 is a schematic diagram showing EMR of a micro-beam focused to a focal point.

FIG. 5 is a graphical representation of the distribution of power density as a function of the distance along a diameter of a focal point.

FIG. 6 is a cross-sectional view of a portion of tissue containing an array of photoselective islets created in a lesion in the tissue.

FIG. 7 is a cross-sectional view of a portion of tissue containing another array of photoselective islets created in a lesion in the tissue.

FIG. 8 is a cross-sectional view of a portion of tissue containing an array of photoselective islets created in a vein of the tissue.

FIGS. 9 and 10 are perspective and side views respectively of a section of a patient's skin and of equipment positioned thereon for practicing one embodiment.

FIGS. 11 and 12 are top views of various matrix arrays of cylindrical lenses, some of which are suitable for providing a line focus for a plurality of target portions.

FIG. 13 is a side schematic view of a handpiece suitable for creating photoselective islets.

FIG. 14 is a cross-sectional view of the handpiece of FIG. 13 through the plane “A” denoted in FIG. 13.

FIGS. 15-19 are cross-sectional views of various exemplary alternative patterns for an array of photoselective islets or other EMR-treated islets.

FIG. 20 is a schematic diagram of a micro-beam focused on a tissue surface.

FIG. 21 is a schematic diagram of a micro-beam focused on a tissue surface and through an optical window to flatten the surface.

FIG. 22 is a schematic diagram of an alternate optical system for generating micro-beams.

FIGS. 23-28 are graphical depictions of simulations of a device that creates photoselective islets and a standard treatment device.

### DETAILED DESCRIPTION

### I. Types of EMR-Treated Islets

When using electromagnetic radiation (“EMR”) to treat tissues, there are substantial advantages to producing lattices of EMR-treated islets in the tissue rather than large, continuous regions of EMR-treated tissue. The lattices are periodic patterns of islets in one, two or three dimensions in which the islets correspond to local maxima of EMR-treatment of tissue. The islets are separated from each other by non-treated tissue (or differently- or less-treated tissue). The EMR-treatment results in a lattice of EMR-treated islets which have been exposed to a particular wavelength, wavelength range, waveband, or spectrum of EMR (or combinations thereof) and which is referred to herein as a lattice of treated islets.

There are many advantages to forming EMR-treated islets, depending on the application. For example, procedures involving EMR-treated islets tend to be more efficacious. The EMR-treated islets may promote faster healing and other responses of the entire tissue. Large portions of tissue may be left undamaged, resulting in less faster recovery. For procedures involving the skin, the epidermis is largely undamaged. The EMR-treated islets can reach deeper tissues more effectively. The spacing of the EMR-treated islets can be selected to optimize the recovery of tissue and other aspects of a procedure. The advantages to forming EMR-treated islets in tissue vary with the application. The various embodiments may have one or more these advantages or may have other advantages entirely.

When the absorption of EMR energy results in significant temperature elevation in the EMR-treated islets, the lattice is referred to herein as a lattice of “thermal islets.” When an amount of energy is absorbed that is sufficient to significantly disrupt cellular or intercellular structures, the lattice is referred to herein as a lattice of “damage islets.” When an amount of energy (usually at a particular wavelength) sufficient to initiate a certain photochemical reaction is delivered, the lattice is referred to herein as a lattice of “photochemical islets.” When an amount or energy is absorbed that is sufficient to ablate the tissue being treated, the lattice is referred to herein as a lattice of “ablated islets.” Conceptually, ablated islets can also be considered a damage islet in which the damage is of a higher degree or is more extreme. When the ablated islets are sufficiently small, for example, on the order of approximately 2 mm or less, the islets are also referred to herein as a lattice of “micro-holes.” When EMR is absorbed selectively by a chromophore, structure or tissue type such that a thermal, damage or ablation islet is created while other surrounding tissue is not affected or is affected to a different degree, the resulting islet is a photoselective islet (or a “selective islet” or “energy selective islet” in cases where the islet is created by non-EMR energy).

By producing EMR-treated islets rather than continuous and/or uniform regions of EMR-treatment, more EMR energy can be delivered to an islet and the risk of bulk tissue damage can be lowered.

Although the methods, devices, systems and other embodiments are generally described in detail for dermatological applications, they can be used for treatment of any tissue surface or subsurface areas to which EMR can be delivered. They can also be used, as described in greater detail below, in a wide range of applications such as drug delivery and the application of fillers to tissue. Additionally, while the methods, devices, systems and other embodiments are generally described using EMR, other types of energy can be used to create such islets, including, without limitation, ultrasound.

A. Lattices of Treated Tissue Generally

EMR-treated islets can also be formed within an area or volume of treated tissue, for example, where the entire tissue area and/or volume is treated with a relatively lower intensity of EMR having a same or different wavelength while the islets are formed by treating portions of the area and/or volume using EMR having a higher intensity. One skilled in the art will recognize that many combinations of parameters are possible that will result in such local maxima of EMR-treatment within the tissue.

When using EMR to treat tissues, whether for purposes of photodynamic therapy, photobiomodulation, photobiostimulation, photobiosuspension, thermal stimulation, thermal coagulation, thermal ablation or other applications, there are substantial advantages to producing lattices of EMR-treated islets in the tissue rather than large, continuous regions of EMR-treated tissue. The EMR-treated tissues can be any hard or soft tissues for which such treatment is useful and appropriate, including but not limited to dermal tissues, mucosal tissues (e.g., oral mucosa, gastrointestinal mucosa), ophthalmic tissues (e.g., retinal tissues), neuronal tissue, vaginal tissue, glandular tissues (e.g., prostate tissue), internal organs, bones, teeth, muscle tissue, blood vessels, tendons and ligaments.

1. Skin Structure

Although the methods, devices and systems described herein can be practiced with many tissues of the body, currently the most common applications of EMR-treatment to tissues are in the field of dermatology. Therefore, the structure of the skin, including its constituent tissues, is described below in some detail, and the remainder of the disclosure will use the skin as an example. In addition, certain applications will be described which are uniquely adapted to the skin (e.g., tattoo removal and permeation of the stratum corneum). It should be understood, however, that the general methods are applicable to other tissues, and that one of ordinary skill in the art can adapt the teachings of the disclosure to other organs and tissues with merely routine experimentation.

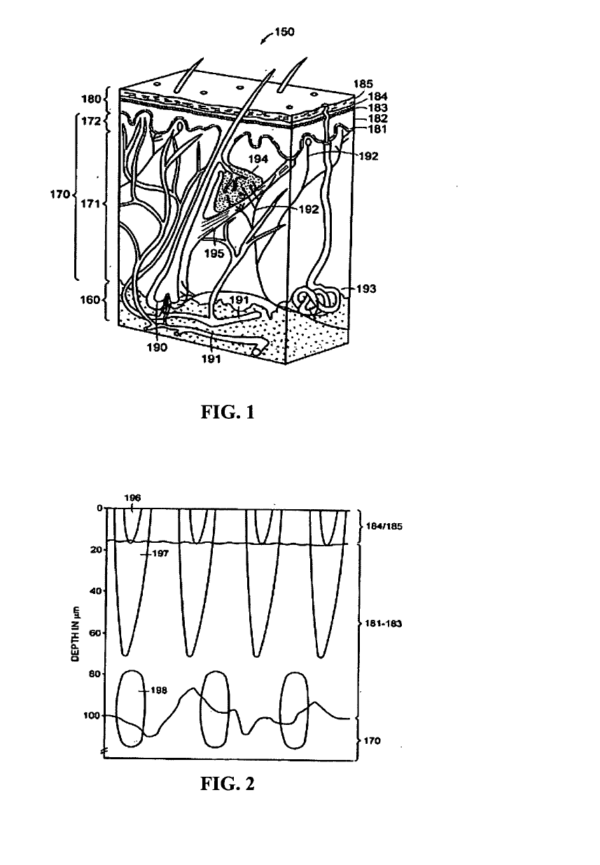

A volume of tissue contains many different structures. The skin is the largest organ in the human body, consisting of several layers of distinct tissues with distinct properties, and ranges in thickness from approximately 0.5 mm to approximately 4 mm. FIG. 1 illustrates a typical cross section of skin 150, showing various layers with differing cellular and intercellular structures.

The skin lies on top of the superficial fascia or subcutaneous tissue 160, a layer of fatty tissue that overlies the more densely fibrous fascia.

Above the subcutaneous tissue is the dermis 170, which comprises fibroelastic connective tissue, and ranges in thickness from approximately 0.3 mm on the eyelids to approximately 3.0 mm on the back. The dermis is highly vascularized and includes a variety of sensory nerves with temperature, pressure and pain receptors that are organized into small nerve bundles that ascend along with the blood vessels and lymphatic vessels to form a network of interlacing nerves beneath the epidermis, i.e., the superficial nerve plexus of the papillary dermis. The dermis includes two layers: a reticular layer 171 and a papillary layer 172. The reticular layer 171 includes cells in a matrix of dense, coarse bundles of collagenous fibers. The papillary layer 172 includes cells in a matrix of loose collagenous and elastic fibers, with elevations or papillae which project toward the epidermis. Cell types in the dermis include fibroblasts, mast cells and macrophages.

The epidermis 180 comprises the outermost stratified layers of the skin, and ranges in thickness from approximately 0.05 mm on the eyelids to approximately 1.5 mm on the palms and soles. The epidermis is avascular and consists largely of epithelial cells which mature as they pass from the innermost layer of columnar cells to the outermost layer of tile-like squamous cells, with the cells becoming increasingly flattened and keratinized as they progress outward. The innermost layer is referred to as the stratum basale, basal cell layer, or stratum germinativum 181, and is the only layer in normal epidermis in which cell division occurs. The next layer, the stratum spinosum 182, includes prickle cells and keratinocytes, and begins the production of keratin. The next layer, the stratum granulosum 183, is a darker layer with intercellular granules and increased keratin production. In thick skin, there is an additional transitional layer, the stratum lucidum 184. Finally, the outermost layer is the stratum corneum (SC) 185, a horny layer of highly keratinized squamous cells.

The cells of the stratum corneum 185 (and the stratum lucidum 184, when present) are highly keratinized (“horny”) and surrounded by an extracellular matrix consisting largely of crystalline lipids. As a result, the stratum corneum forms a hard, resilient barrier to water transport, and is not permeable to most aqueous or organic solvents or solutes. The stratum corneum 185 is about 15 μm deep on most anatomic sites but can be in the ranges of 10-300 μm (e.g., 20 μm at the forearm and 50-60 μm at the wrist).

Also shown are typical organs and structures within the skin, including a hair follicle 190, blood vessels 191, nerve fibers 192, a sweat gland 193, a sebaceous gland 194, and an arrector pili muscle 195. Normal skin temperature is approximately 29-37° C. When exposed to temperatures in excess of 40-43° C., the sensory nerves of the dermis will transmit a pain response in most human subjects.

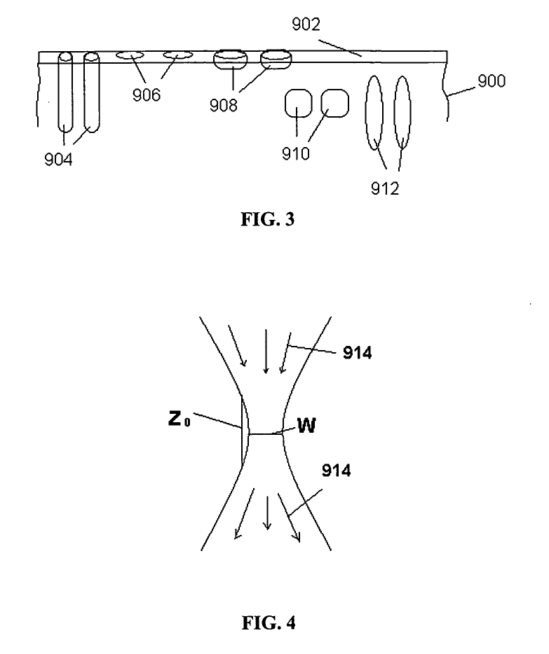

FIG. 2 is a schematic cross-sectional view of a human skin section 150. It shows depths into the skin, from the surface in μm. The stratum corneum 185 and stratum lucidum 184 are shown extending to a depth of approximately 15 μm below the skin surface. The remaining layers (i.e., layers 181-183) of the epidermis 180 extend from the stratum lucidum/corneum 184/185 to the boundary with the dermis 170 at a depth from the surface in the range of approximately 50-150 μm. Also shown are exemplary shallow islets 196 affecting the stratum lucidum/corneum 184/185, deeper islets 197 affecting the stratum lucidum/corneum 184/185 and deeper layers of the epidermis 180, and subsurface islets 198 spanning portions of the deeper epidermis 180 and upper dermis 170.

The depths shown in FIG. 2 are merely exemplary. Different locations in the typical human body have different depth profiles for the stratum corneum/lucidum, epidermis, and dermis. In addition, as described below. a great variety of other islet configurations are possible which are not shown in the figure (e.g., islets entirely in the dermis; islets entirely in the subcutaneous tissue; islets spanning the dermis and subcutaneous tissue; islets spanning portions of the epidermis, dermis and subcutaneous tissue).

2. Lattices of EMR-Treated Islets In Skin and Other Tissue

The lattices are periodic patterns of islets in one, two or three dimensions in which the islets correspond to local maxima of EMR-treatment of tissue. The islets are separated from each other by non-treated tissue (or differently- or less-treated tissue). The EMR-treatment results in a lattice of EMR-treated islets which have been exposed to a particular wavelength or spectrum of EMR, and which is referred to herein as a lattice of “optical islets.” When the absorption of EMR energy results in significant temperature elevation in the EMR-treated islets, the lattice is referred to herein as a lattice of “thermal islets.” When an amount of energy is absorbed that is sufficient to significantly disrupt cellular or intercellular structures, the lattice is referred to herein as a lattice of “damage islets.” When an amount of energy (usually at a particular wavelength) sufficient to initiate a certain photochemical reaction is delivered, the lattice is referred to herein as a lattice of “photochemical islets.”

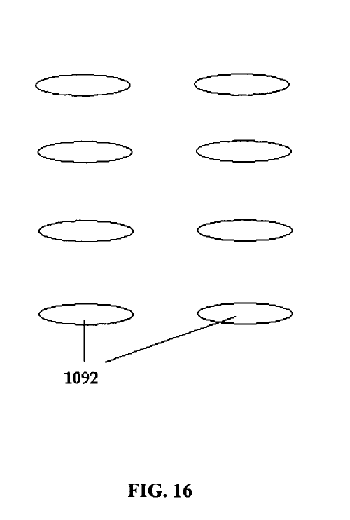

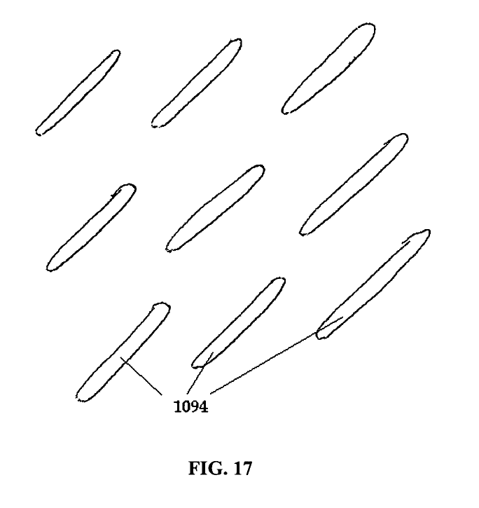

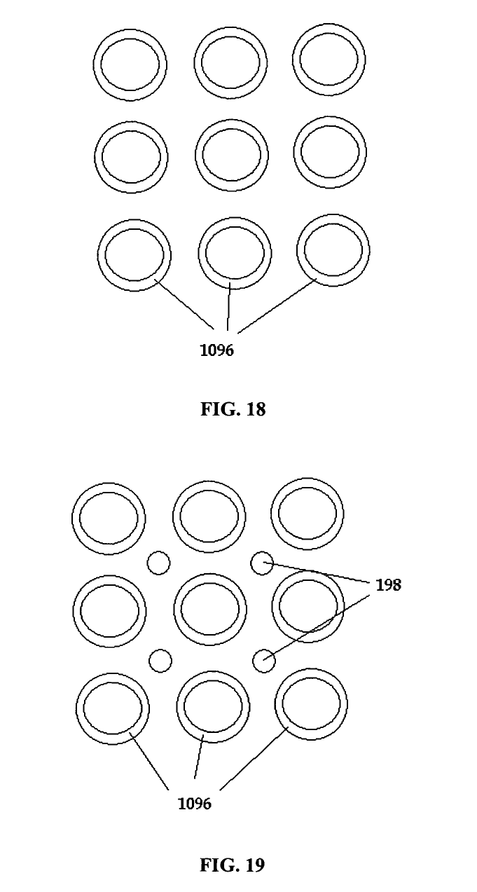

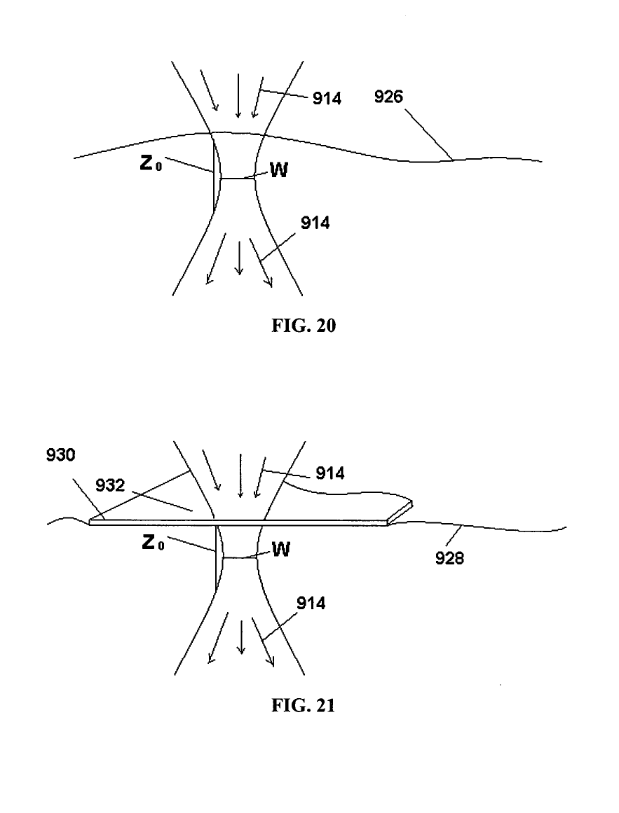

By producing EMR-treated islets rather than continuous regions of EMR-treatment, untreated regions (or differently- or less-treated regions) surrounding the islets can act as thermal energy sinks, reducing the elevation of temperature within the EMR-treated islets and/or allowing more EMR energy to be delivered to an islet without producing a thermal islet or damage islet and/or lowering the risk of bulk tissue damage. Moreover, with respect to damage islets, it should be noted that the regenerative and repair responses of the body occur at wound margins (i.e., the boundary surfaces between damaged and intact areas) and, therefore, healing of damaged tissues is more effective with smaller damage islets, for which the ratio of the wound margin to volume is greater. Furthermore, as discussed in conjunction with FIGS. 15-19, by altering the shape of the islets, the ratio of surface area of the islet to the volume of the islet can be increased, which will also improve the effective healing response.

As described more fully below, the percentage of tissue volume which is EMR-treated versus untreated (or differently- or less-treated) can determine whether optical islets become thermal islets, damage islets, ablation islets or photochemical islets. This percentage is referred to as the “fill factor”, and can be decreased by increasing the center-to-center distance(s) (i.e., pitch) of EMR-treated islets of fixed volume(s), and/or decreasing the volume(s) of islets of fixed center-to-center distance(s).

Because untreated tissue volumes act as a thermal sink, these volumes can absorb energy from treated volumes without themselves becoming thermal or damage islets. Thus, a relatively low fill factor can allow for the delivery of high fluence energy to some volumes while preventing the development of bulk tissue damage. Finally, because the untreated tissue volumes act as a thermal sink, as the fill factor decreases, the likelihood of optical islets reaching threshold temperatures to produce thermal islets or damage islets also decreases (even if the EMR power density and total exposure remain constant for the islet areas).

Multiple EMR-treated volumes of the skin can be created that are separated by untreated volumes. The multiplicity of volumes can be described as defining a “lattice,” and the treated volumes, because they are separated by untreated volumes, can be described as “islets” within the skin. As used herein, the terms “treatment islet,” “islets of treatment,” and “EMR-treated islets” are used interchangeably to refer to any of the categories of islets described below.

EMR-treatment of completely or partially isolated volumes or islets of tissue produces a lattice of EMR-treated islets surrounded by untreated volumes. Islets can be treated with any form of EMR, as many embodiments include the use of EMR within the ultraviolet, visible and infra-red spectrum. Other forms of EMR that are useful include microwave, ultrasound, photo-acoustic, radio frequency, low frequency and EMR induced by direct current.

As noted above, when the total energy transfer per unit cross-sectional area (i.e., fluence) or the rate of energy transfer per unit cross-sectional area (i.e., flux) becomes sufficiently high, the tissue of an optical islet will be heated, resulting in a thermal islet. If the temperature increase is sufficiently high, the tissue of a thermal islet will be damaged, resulting in a damage islet. In some embodiments, as described below, it can be desirable to produce islets without producing thermal or damage islets. In such embodiments, the fill factor can be decreased in order to provide a greater volume of untreated tissue to act as a thermal sink.

B. Thermal Islets

EMR-treatment of isolated volumes or islets of tissue can produce a lattice of thermal islets with temperatures elevated relative to those of surrounding untreated volumes. Thermal islets result when energy is absorbed by an EMR-treated islet significantly faster than it is dissipated and, therefore, significant heating occurs.

Heating can result from the absorbance of EMR by water present throughout a volume of treated tissue, by endogenous chromophores present in selected cells or tissue(s) (e.g., melanin, hemoglobin), by exogenous chromophores within the tissue (e.g., tattoo ink) or, as described below, by exogenous chromophores applied to the surface of the tissue.

With respect to skin, in order to avoid causing pain to a subject, the maximal temperature of the basal membrane, which is adjacent to the nerve terminals of the papillary dermis, should not exceed 40-45° C. Assuming no active cooling of the skin surface, the temperature rise in the basal membrane, ΔT2, can be related to the temperature rise in the hyperthermic islets, ΔT1, by an approximate formula:

ΔT2=fΔT1

where f is the fill-factor of the optical lattice at the skin surface. This formula indicates that the temperature in the SC can attain relatively high values without triggering the pain response of the body if the fill factor is sufficiently low.

For example, setting ΔT2 to 12° C. and f to 0.3 yields ΔT1 of 40° C. In practice, the temperature rise ΔT1 may be limited by other factors, such as, for example, the threshold of structural damage to the SC or the desired size of the damage islets.

The thermal islet model is based, in part, on the time-dependent heat equation. Specifically, as described in more detail below, the thermal constants of the skin layers are obtained from Takata's relations (Takata et al. (1977), in Report SAM-TR-77-38 (San Antonio, Tex.: US Air Force School for Aerospace Medicine)) and are functions of the volume fraction of water in the corresponding layer. Specific effects associated with the bio-heat equation, e.g., the metabolic heat generation and the change of the blood perfusion rate while heating the living tissue, can be neglected for EMR pulses of short duration (Sekins et al. (1990), Thermal Science for Physical Medicine, in Therapeutic Heat and Cold, 4th edition, Lehmann, ed. (Baltimore: Williams & Wilkins) pp. 62-112). In practice, the EMR-heating can dominate strongly over metabolic heating and heat transfer by the blood flow. Moreover, the changes in the blood perfusion rate can occur with the delay of about 1 min with respect to the variations of the tissue temperature (Sekins et al. (1990), in Therapeutic Heat and Cold, 4th edition, Lehmann, ed. (Baltimore: Williams & Wilkins) pp. 62-112), and do not affect the islet formation dynamics unless tissues are under combined action (with EMR) of simultaneous physical factors (e.g., elevated or lowered external pressure, ultrasound, elevated or lowered skin surface temperature).

A lattice of thermal islets is a time-dependent phenomenon. If absorptive heating occurs at too great a rate or for too long a period, heat will begin to diffuse away from the EMR-treated islets and into the surrounding untreated tissue volumes. As this occurs, the thermal islets will spread into the untreated (or lesser-treated or differently-treated) volumes and, ultimately, the thermal islets will merge and result in bulk heating. By using a sufficiently short pulse width relative to the temperature relaxation time of the target, it is possible to avoid merging or overlapping of thermal islets in a lattice.

C. Damage Islets

EMR-treatment of isolated volumes or islets of tissue can produce a lattice of damage islets surrounded by volumes of undamaged tissue (or differently- or less-damaged tissue). Damage islets result when the temperature increase of an EMR-treated thermal islet is sufficient to result in protein coagulation, thermal injury, photodisruption, photoablation, or water vaporization. Depending upon the intended use, damage islets with lesser degrees of damage (e.g., protein coagulation, thermal injury) or greater degrees of damage (e.g., photodisruption, photoablation, or water vaporization) may be appropriate. As before, damage can result from the absorbance of EMR by several mechanisms, including water present throughout a volume of treated skin, by endogenous chromophores present in selected cells or tissue(s) in the skin (e.g., melanin, hemoglobin), by exogenous chromophores within the skin (e.g., tattoo ink) or, as described below, by exogenous chromophores applied to the surface of the skin.

In some embodiments, the damage islets are thermal injuries with coagulation of structural proteins. Such damage can result when, for example, the light pulse duration varies from several microseconds to about 1 sec, but the peak tissue temperature remains below the vaporization threshold of water in the tissue (Pearce et al. (1995), in Optical-Thermal Response of Laser-Irradiated Tissue, Welch et al., eds. (Plenum Press, New York), pp. 561-606). The degree of damage is a function of the tissue temperature and the duration of the thermal pulse, and can be quantified by any of several damage functions known in the art. In the description below, for example, the damage function yielding the Arrhenius damage integral (Pearce et al. (1995), in Optical-Thermal Response of Laser-Irradiated Tissue, Welch et al., eds. (Plenum Press, New York), pp. 561-606; Henriques (1947), Arch. Pathol. 43:480-502) is employed. Other mechanisms and models of damage islet formation can apply to embodiments with relatively short and intense pulses, particularly in connection with photodisruption, photoablation, and water vaporization.

D. Photochemical Islets

In accordance with another aspect, EMR-treatment of isolated volumes or islets of tissue can produce a lattice of photochemical islets surrounded by volumes of tissue in which a photochemical reaction has not been induced. The photochemical reaction can involve endogenous biomolecules or exogenous molecules. For example, exposure of the skin to certain wavelengths of EMR can result in increased melanin production and “tanning” through the activation of endogenous biomolecules and biological pathways. Alternatively, for example, exogenous molecules can be administered in photodynamic therapy, and activation of these molecules by certain wavelengths of EMR can cause a systemic or localized therapeutic effect.

E. Micro-Holes and Ablation Islets

One specific form of EMR-treated islets are volumes in which the tissue has been damaged, ablated or otherwise treated to form small holes, channels, openings, chambers and/or similar structures in the tissue. (For convenience, such structures are referred to collectively as micro-holes.) The damage to the tissue in the islet is to the degree that the tissue is vacated to form empty space or is altered in composition, such as, for example, in the case of a channel of tissue that is damaged such that the channel is vacated or primarily filled with water, other fluid and/or remnants or vestiges of the damaged tissue (e.g., tissue fibers or other substances).

Micro-holes can be used for a variety of purposes such as, for example, the application of drugs and medicines, the injection of fillers and other inert substances, and the removal of fat tissue or other substances. Micro-holes can be used as channels for the local delivery of the desirable therapeutical compound(s) to the target (treated) anatomical areas by diffusion or by employing but not limited to the other approaches, such as vesicle/particle transporters, by physical, chemical or electrical manipulations (for instance electroporation, iontoporation, sonophoresis, magnetophoresis, photomechanical waves, niosomes, transfersomes etc.). Micro-holes can be created in any tissue, such as skin, nail, bone, muscle, etc., and at any anatomical location.

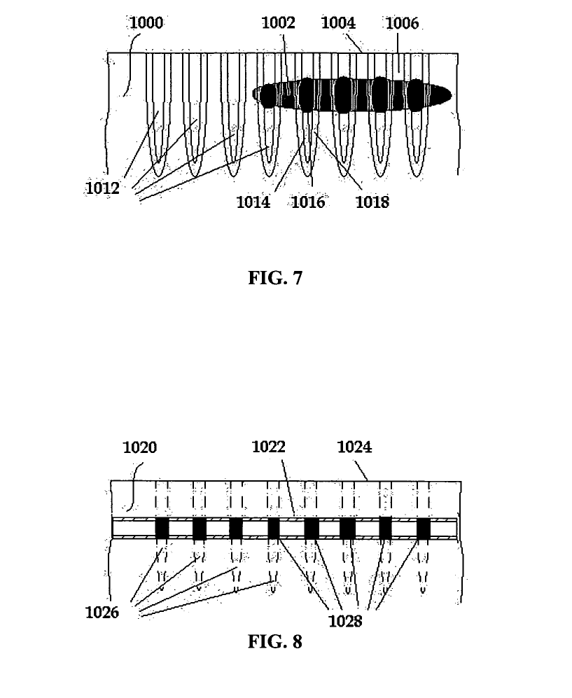

Referring to FIG. 3, examples of various micro-hole structures are shown. Micro-holes 904 are channels extending from a surface 902 of tissue 900 and into the tissue 900. Micro-holes 906 are openings that lie at the surface 902 of the tissue, but that do not extend deeply into the tissue. Micro-holes 908 are depressions that lie at the surface 902, but that extend slightly into the tissue 900 to a greater depth than micro-holes 906. Micro-holes 910 are chambers within the tissue 900 and below the surface 902. Similarly, micro-holes 912 are chambers that are elongated to form columns but that do not have an opening through the surface. The micro-holes shown in FIG. 3 are simplified for purposes of the above description. Depending on how a micro-hole is formed, its structure is likely more complex. For example, a micro-hole formed by ablating tissue may have a zone or halo of damage surrounding the vacated hole. This is shown more clearly in conjunction with FIG. 7 discussed below. (Note also, that similar structures corresponding to the structures shown in FIG. 3 can be created for other types of islets, such as thermal islets and other damage islets.)

Micro-holes can be various sizes, including, without limitation, micro-holes that are macroscopic or microscopic in size. For example, a lattice of micro-holes on nail tissue can have a diameter of 50 micrometers, but much smaller micro-holes are possible. Referring to FIGS. 4 and 5, the size of a micro-hole is determined essentially by the spot size at which EMR is applied to the tissue, the power density of the EMR that is applied, the pulse duration of the EMR, the wavelength of EMR that is applied and the threshold of ablation in the tissue that is irradiated (or other thresholds, for example, the threshold of thermal damage in other embodiments). To maximize the intensity of the radiation, the spot size of a micro-hole is preferably the diameter of the focal point. Using currently available optics, therefore, micro-holes can be formed having a diameter of approximately 0.1×λ (i.e., 10% of the wavelength of the applied radiation). However, even smaller diameters are theoretically possible, depending on the quality of optics and the design of optics that are used.

The spot size that can be created (and, thus, the resulting micro-hole) is proportional to the wavelength: the smaller the wavelength, the smaller the micro-hole that can be created. FIG. 4 shows a focused beam of rays 914 of EMR in which the focal point has a diameter W greater than the wavelength of the EMR. Theoretically, the smallest spot size that is possible for an individual EMR beam is the smallest focal point that can be achieved. The smallest focal point that may be achieved has a diameter (W) that is approximately the wavelength (λ) of the EMR that is applied. (If non-coherent light is applied, the smallest spot size that is theoretically possible is the largest wavelength among the effective wavelengths that are applied to treat the tissue. For example, if one or more spectral bands of EMR are applied to the tissue, but only a subset, subsets, or sub-band(s) of the EMR are actually used to ablate or otherwise treat and form the islet, the smallest possible diameter of the resulting micro-hole will be the size of the largest wavelength in the sub-band(s) or subset(s) of EMR.)

The focal depth (Z₀) of the spot size is a function of the diameter of the focal point, which is determined by the following equation:

$${Z}_{0} = \frac{π * {W}^{2}}{λ}$$

Thus, in an example where the focal point has a diameter of 30 μm and the wavelength is 3 μm, the focal depth is approximately 943 μm.

FIG. 5 shows the power density of EMR as a function of distance across the focal point of the applied EMR. In the case shown, the EMR has been focused to a point having a diameter equal to the wavelength of the applied EMR. When EMR is applied to tissue, the power of the EMR has a roughly Gaussian distribution with the highest intensity at the mid-point of the focal point, in this case, the midpoint of the wavelength of EMR used. When the power of the applied EMR exceeds the threshold of ablation, a micro-hole is formed.

As seen in FIG. 5, the size of the holes 922 and 924 can be controlled by adjusting the power applied. In the example illustrated, a power distribution 916 exceeds a threshold of ablation 918 for approximately one-half of the focal point, i.e., the wavelength λ. However, when the power of the applied EMR is reduced, a power distribution 920 exceeds the threshold of ablation 918 over a much smaller portion of the focal point: in the case shown, approximately 0.1× the diameter of the focal point or approximately 0.1×λ. Theoretically, the micro-holes could be any non-zero number, but practically other factors may provide a lower limit to the size of the diameter of the micro-holes.

In other embodiments, the power density may be modulated during the formation of a single micro-hole. For example, a first pulse of EMR can be applied at a first power density and a second pulse can be applied at a different power density. If the power densities of multiple pulses are alternated in this fashion, micro-holes having varying diameters can be formed. Such micro-holes may have various benefits, for example, an increase in surface area that can be used to deliver substances such as drugs or clearing substances more effectively or at a faster rate. Similarly, the power density can be modulated, for example, between pulses, during pulses or during the application of EMR in a continuous or quasi-continuous wave, to form micro-holes of varying shapes, such as, for example an conical-like shape. A conical shape in which the narrow portion of the cone is at the surface of the tissue and in which the base of the cone lies within the tissue could be used to create a micro-hole having a relatively larger volume, which can be used, for example, to hold a substance, and also having a relatively small opening, which will close more quickly than a larger hole.

When using ablation to form a micro-hole, the ablation is preferably performed in conjunction with a device to remove the ablated material, although this is not required. When tissue is ablated, remnants of the tissue generally remain in the micro-holes. This can increase the amount of refraction and otherwise decrease optimum performance of the device forming the micro-holes. The micro-holes are formed more precisely when the ablated material is removed. There are many embodiments possible of a system, device or method to remove tissue, such as, for example, a device that is synchronized to produce a short pulse of air at high pressure, which expels the ablated material immediately after a pulse of EMR is applied before the material has a chance to settle in the micro-hole that is being formed. Many different embodiments are possible for removing tissue. For example, devices in which the EMR is delivered through an optical element such as a lens that is not in contact with the tissue can include a device that directs air or other gas into the space between the tissue and the optical element to remove the remnants of the ablated tissue. In embodiments where an optical element from which EMR is delivered is in contact with the tissue, other structures can be used. For example, the optical element may contain ribs, ridges, channels or other structures through which a high-pressure gas may be pulsed such that the remnants of ablated tissue are removed through those structures as the device is moved relative to the tissue during operation. Similarly, in still other embodiments, some or all of the remnants of the ablated tissue can be left within the micro-holes. However, if tissue is ablated and not subsequently vacated from the EMR-islet, additional factors will affect the characteristics of the resulting micro-hole. For example, scattering within the tissue, including the remnants of the ablated tissue, may increase and impact the size, shape and other characteristics of the micro-hole.

While the above has been discussed in terms of the threshold of ablation, the concept can be applied similarly to other types of EMR-Islets, for example, by using thresholds of damage instead of thresholds of ablation. Non-ablative techniques may be used to form similar micro-structures, such as thermal islets, damage islets, zones of thermally damaged tissue or small zones of healthy tissue surrounded by zones of EMR-treated tissue, such as, for example, thermally treated tissue and/or ablated tissue.

F. Photoselective Islets

EMR-treated islets can also be created by taking advantage of the photoselective nature of different types of tissue and/or different types of chromophores. Such islets can take various forms, including thermal islets, damage islets, micro-holes, ablation islets, and other structures and types of damage (e.g., small or micro zones of thermal damage located at a depth below the surface of the tissue).

Although photoselective islets and other energy selective islets can be created in many different tissues, such as muscle, tendon, bone and gastrointestinal tissue, the treatment of skin is used as an exemplary embodiment. The various structures in the skin absorb EMR to varying degrees. The photoselective vaporization of tissue, such as tissue subject of removal for treatment of gastrointestinal, glandular and gynecological conditions, is based upon applying a high intensity radiation to tissue using a radiation that is highly absorptive in the tissue, while being absorbed only to a negligible degree by water or other irrigant during the operation, at power densities such that the majority of the energy is converted to vaporization of the tissue without significant residual coagulation of adjacent tissue.

The selective absorption of EMR by different types of tissue results in the creation of islets, such as thermal or damage islets, in specific targets in such a manner that the temperature of the surrounding tissue is maintained below the threshold for the creation of such islets. The selectivity is obtained by selection of a proper wavelength and pulse duration. For example, the presence of melanin in the epidermal layer can be used to create EMR-islets within the portion of the tissue containing a high concentration of melanin.

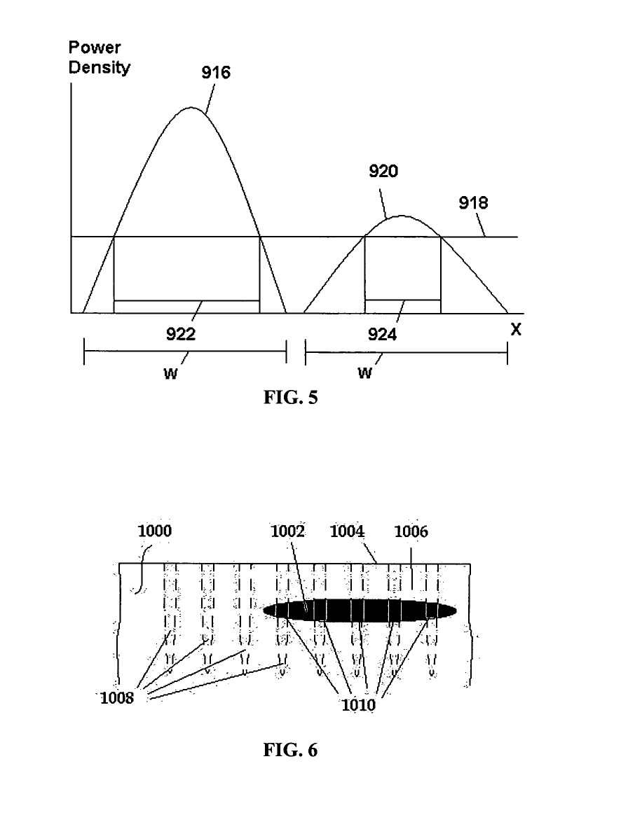

Referring to FIG. 6, skin tissue 1000 contains a volume 1002, which is a pigmented lesion containing a high concentration of melanin. Volume 1002 is located below a surface 1004 of skin tissue 1000, and is surrounded by surrounding skin tissue 1006 containing less melanin. The melanin is treated using a fractional device that creates EMR-treated islets within the volume 1002, without creating such islets within the surrounding tissue 1006.

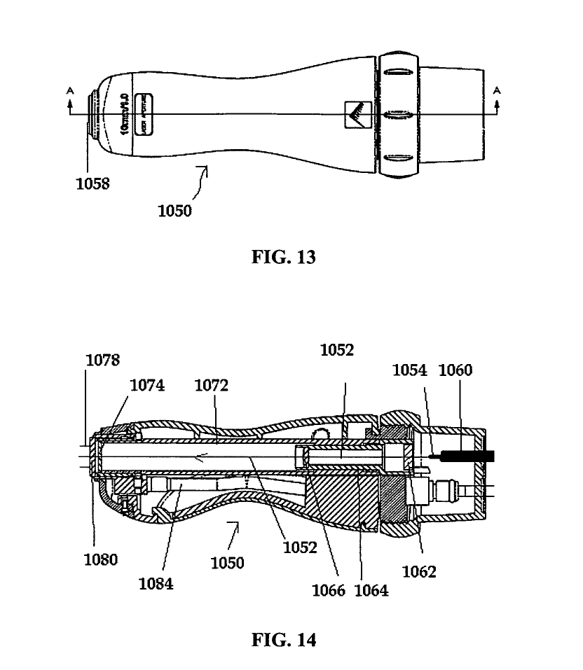

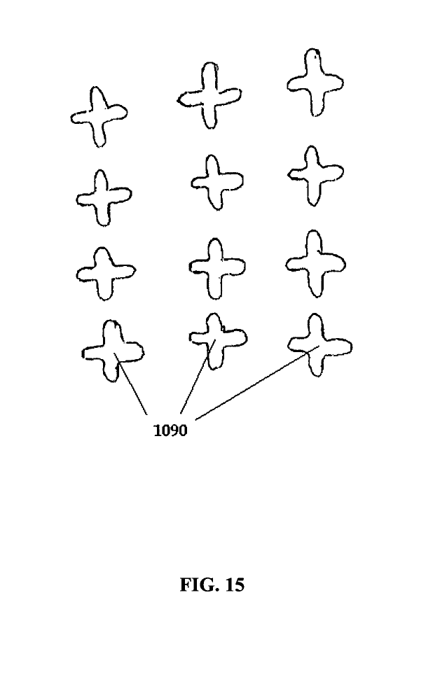

In one embodiment, micro-beams of EMR are applied to the surface of the tissue 1000. The tissue 1000 is irradiated with columns of EMR 1008 having a wavelength of 1064 nm, using a device similar to that described in conjunction with FIGS. 13 and 14. The power density of the individual micro-beams is selected to prevent any damage to the surrounding tissue 1006. Thus, EMR passes through the surrounding tissue 1006, but, due to the relatively lower coefficient of absorption of this tissue, there is no damage to the tissue and no EMR-islets are created in the surrounding tissue 1006. However, due to the relatively higher coefficient of absorption of the melanin within volume 1002, considerably more EMR is absorbed at the intersections 1010 of EMR columns 1008 and volume 1002. Thus, the tissue at intersections 1010 is damaged and EMR islets are formed at those locations.

In the embodiment described, no EMR-islet is formed except at intersections 1010. However, in other embodiments, other combinations are possible by adjusting the parameters of the EMR that is applied. For example, if EMR is applied at a relatively higher power density, EMR-islets can be created at intersections 1010 and in EMR columns 1008. In another embodiment, damage islets can be created at intersections 1010, while thermal islets are created in the remainder of EMR columns 1008 (i.e., the portions of the columns with relatively low level of melanin or other chromophore).

Referring to FIG. 7, in yet another embodiment, the power density of the EMR micro-beams is selected to create an array of damage islets extending in a column from the surface of the tissue and through the melanin in the tissue. In this embodiment, damage islets 1012 are created throughout the column of tissue in which the EMR is applied. Damage islets 1012 have two or three chief components depending on their location: (1) a column of damage 1014; (2) a volume of extreme damage 1016 (which occur within volume 1002 and not in the columns that lie without volume 1002); and (3) a surrounding halo of thermal damage 1018. Columns of damage 1014 are approximately coextensive with the EMR columns 1008 shown in FIG. 6. The volumes of extreme damage 1016 are approximately coextensive with intersections 1010 shown in FIG. 6. However, due to the higher intensity of EMR and the high coefficient of absorption of the melanin, the actual boundaries of the volume of extreme damage 1016 exceed the boundaries of intersections 1010. The halos of thermal damage 1018 surround damage islets 1012, and represent a zone in which heat accumulated to a degree sufficient to create a thermal islet, without causing damage to the tissue, such as coagulation.

The heating mechanism for time scales less than typically 1 microsecond(s) corresponds to a transient local heating of the individual melanosomes. For larger time scales, heat diffusion out of the melanosomes become of increased importance, and the temperature distribution will reach a local steady state condition after typically 10 microsecond(s). For longer pulse durations, heat diffusing from neighboring melanosomes becomes important, and the temperature rise in a time scale from 100-500 microsecond(s) is dominated by this mechanism.

One skilled in the art will appreciate that the various dimensions and characteristics of the various components of the EMR-islets can be controlled by altering the various parameters of the EMR that is applied, such as wavelength, fluence, power density, pulse duration, pulse shape, etc. For example, the EMR can be applied at an intensity sufficient to ablate the volumes of extreme damage 1016. (Although, in this example, the remains of the ablated tissue are contained within the volume of extreme damage 1016 until extricated from the tissue by biological or other processes.) Similarly, the surrounding halo of thermal EMR-islets can be relatively large compared to the damaged volume, if, for example, a high intensity of EMR is applied in a relatively longer pulse such that more heat accumulates in the surrounding tissue. Similarly, if a shorter pulse and/or lower intensity are used, the surrounding halo may be small or essentially non-existent. The amount of relative damage between the columns of damage 1014 and the volumes of extreme damage 1016 can be adjusted by shifting the wavelength of EMR applied or by applying multiple wavelengths, such that the relative amounts of absorption of EMR in columns 1014 and volumes 1016 can be more even or more disparate, depending on the application.

Chromophores other than melanin may also be targeted. For example, blood can be targeted as a chromophore to cause damage to the portions of a blood vessel that directly surround the targeted volumes. Such a treatment can be used, for example, to treat vascular lesions (such as port wine stains), leg veins, or for medical purposes in vascular surgeries. As shown in FIG. 8, a volume of tissue 1020 contains a blood vessel 1022 located below a surface 1024 of the tissue 1020. EMR-islets 1028 are formed at the vessel walls due to the heat that is transferred from the chromophore, i.e., the blood in this case, to the wall of the blood vessel. In this embodiment, the EMR-islets are damage islets that damage the vessel wall, but allow the wall to repair itself. In other embodiments, the intensity of the EMR can be increased to destroy portions of the blood vessel and prevent perfusion of the blood into the area, as in the case of vascular lesions and the destruction of varicose veins. For port wine stains, the wavelength can be in a range of 900 to 1850 nm for water absorption or 380 to 1100 nm for hemoglobin absorption. Still other embodiments are possible. Thus, photoselective islets can be created in various ways in the same tissue by targeting different chromophores using different wavelengths or combinations of wavelengths of EMR.

A different type or degree of islet 1026 may also be formed, for example, a thermal islet in the portions of columns 1026 that are not included in islets 1028. Alternatively, the light penetrating in columns 1026 may be at an intensity, wavelength or other parameter that has no perceived lasting effect on the skin, for example, the temperature of the tissue in those columns may rise but no other effect remains when the temperature returns to normal.

G. Non-Selective Sub-Surface EMR-Islets

As discussed above, EMR-islets can be created within and below the surface of tissue. In addition to creating such islets by selective methods, non-selective methods can be used. Thus, a technique is provided (a) which permits various therapeutic treatments on a patient's body at depths to 4 mm or more, (b) which permits islands of damage in three dimensions to occur, thereby facilitating healing (by permitting continued blood flow and cell proliferation between skin layers and islands of damage 214) and reducing patient discomfort, (c) which permits targeting of specific components for treatment without damage to surrounding parts of the patient's body, thereby more efficiently using the applied radiation while also reducing peripheral damage to the patient's body as the result of such treatment (d) which permits treatment of all skin types using substantially the same parameters for a given treatment, thereby simplifying treatment set-up and treatment safety, and/or (e) which permits the wavelength utilized for treatment to be optimally selected for the depth of treatment, rather than being restricted to a wavelength optimally absorbed by a targeted chromophore.

II. Parameters Associated with EMR-Treated Islets

In practice, a variety of different treatment parameters relating to the applied EMR can be controlled and varied according to the particular cosmetic or medical application. These parameters include, without limitation, the following:

A. The Shape of EMR-Treated Islets

The optical islets can be formed in any shape which can be produced by the devices described below. Thus, depending upon the wavelength(s), temporal characteristics (e.g., continuous versus pulsed delivery), and fluence of the EMR; the geometry, incidence and focusing of the EMR beam; and the index of refraction, absorption coefficient, scattering coefficient, anisotropy factor (the mean cosine of the scattering angle), and the configuration of the tissue layers; and the presence or absence of exogenous chromophores and other substances, the islets can be variously-shaped volumes extending from the surface of the skin through one or more layers, or extending from beneath the surface of the skin through one or more layers, or within a single layer. If the beams are not convergent, such beams will define volumes of substantially constant cross-sectional areas in the plane orthogonal to the beam axis (e.g., cylinders, rectanguloids). Alternatively, the beams can be convergent, defining volumes of decreasing cross-sectional area in the plane orthogonal to the central axis of the beams (e.g., cones, pyramids). The cross-sectional areas can be regular in shape (e.g., ellipses, polygons) or can be arbitrary in shape. In addition, depending upon the wavelength(s) and fluence of an EMR beam, and the absorption and scattering characteristics of a tissue for the wavelength(s), an EMR beam may penetrate to certain depths before being initially or completely absorbed or dissipated and, therefore, an EMR-treated islet may not extend through the entire depth of the skin but, rather, may extend between the surface and a particular depth, or between two depths below the surface.

Generally, the lattice is a periodic structure of islets in one, two, or three dimensions. For instance, a two-dimensional (2D) lattice is periodic in two dimensions and translation invariant or non-periodic in the third. The type of periodicity is characterized by the voxel shape. For example, and without limitation, there can be layer, square, hexagonal or rectangle lattices. The lattice dimensionality can be different from that of an individual islet. A single row of equally spaced infinite cylinders is an example of the 1D lattice of 2D islets (if the cylinders are of finite length this is the 1D lattice of 3D islets). The lattice dimensionality is equal to or smaller than the dimensionality of its islets (this fact follows from the fact that the lattice cannot be periodic in the dimension where its islets are translation invariant). Hence, there exists a total of 6 lattice types with each type being an allowed combination of the islet and lattice dimensionalities. For certain applications, an “inverted” lattice can be employed, in which islets of intact tissue are separated by areas of EMR-treated tissue and the treatment area is a continuous cluster of treated tissue with non treated islands.

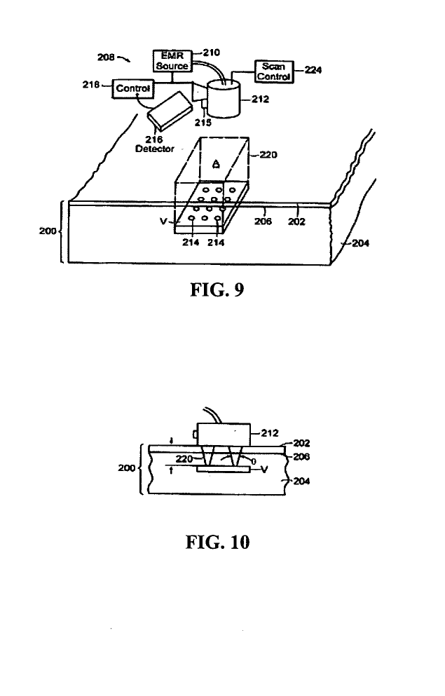

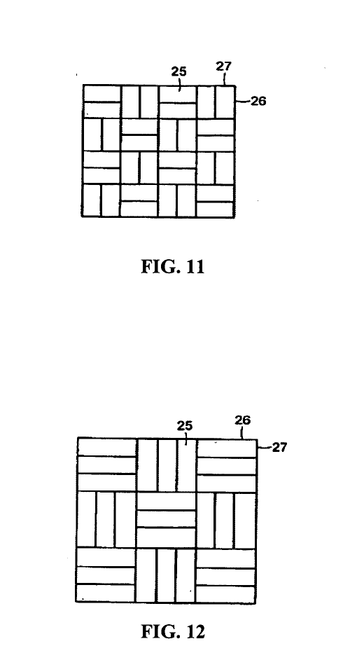

Referring to FIGS. 9 and 10, each of the treated volumes can be a relatively thin disk, as shown, a relatively elongated cylinder (e.g., extending from a first depth to a second depth), or a substantially linear volume having a length which substantially exceeds its width and depth, and which is oriented substantially parallel to the skin surface. The orientation of the lines for the islets 214 in a given application need not all be the same, and some of the lines may, for example, be at right angles to other lines. (See for example FIGS. 11 and 12, showing exemplary lens arrays 27 each having cylindrical lenses 25 and 26 oriented at right angles.) Lines also can be oriented around a treatment target for greater efficacy. For example, the lines can be perpendicular to a vessel or parallel to a wrinkle. Islets 214 can be subsurface volumes, such as spheres, ellipsoids, cubes or rectanguloids of selected thickness. The islets can also be substantially linear or planar volumes. The shapes of the islets are determined by the combined optical parameters of the beam, including beam size, amplitude and phase distribution, the duration of application and, to a lesser extent, the wavelength.

The parameters for obtaining a particular islet shape can be determined empirically. For example, a 1720 nm laser operating with a low conversion beam at approximately 0.005-2 J and a pulse width of 0.5-2 ms, can produce a generally cylindrically shaped islet. Alternatively, a 1200 nm laser operating with a highly converting beam at approximately 0.5-10 J and a pulse width of 0.5-3 sec, can produce a generally ellipsoid-shaped islet.

By suitable control of wavelength, focusing, incident beam size at the surface and other parameters, the islets, regardless of shape, can extend through a volume, can be formed in a single thin layer of a volume, or can be staggered such that adjacent islets are in different thin layers of volume. Most configurations of a lattice of islets can be formed either serially or simultaneously. Lattices with islets in multiple thin layers in a volume can be formed serially, for example using a scanner or using multiple energy sources having different wavelengths. Islets in the same or varying depths can be created, and when viewed from the skin surface, the islets at varying depths can be either spatially separated or overlapping.

The geometry of the islets affects the thermal damage in the treatment region. Since a sphere provides the greatest gradient, and is thus the most spatially confined, it provides the most localized biological damage, and can therefore be preferred for applications where this is desirable.

B. The Size of EMR-Treated Islets

The size of the individual islets within the lattices of EMR-treated islets can vary widely depending upon the intended cosmetic or medical application. As discussed more fully below, in some embodiments it is desirable to cause substantial tissue damage to destroy a structure or region of tissue (e.g., a sebaceous gland, hair follicle, tattooed area) whereas in other embodiments it is desirable to cause little or no damage while administering an effective amount of EMR at a specified wavelength (e.g., photodynamic therapy). As noted above with respect to damage islets, however, the healing of damaged tissues is more effective with smaller damage islets, for which the ratio of the wound margin to volume is greater.

As a general matter, the size of the EMR-treated islets can range, for example, from 1 μm to 30 mm in any particular dimension. For example, and without limitation, a lattice of substantially linear islets can consist of parallel islets have a length of approximately 30 mm and a width of approximately 10 μm to 1 mm. As another example, and without limitation, for substantially cylindrical islets in which the axis of the cylinder is orthogonal to the tissue surface, the depth can be approximately 10 μm to 4 mm and the diameter can be approximately 10 μm to 1 mm. For substantially spherical or ellipsoidal islets, the diameter or major axis can be, for example, and without limitation, approximately 10 μm to 1 mm. Thus, in some embodiments, the islets can have a maximum dimension in the range from 1 μm to 10 μm, 10 μm to 100 μm, 100 μm to 1 mm, 1 mm to 10 mm, or 10 mm to 30 mm, as well as all possible ranges within 1 μm to 30 mm.

When considering the size of the optical, thermal, damage or photochemical islets, it is important to note that the boundaries of the islets may not be clearly demarcated but, rather, may vary continuously or blend into the untreated tissue (or differently- or less-treated tissue). For example, EMR beams are subject to scattering in various tissues and, therefore, even beams of coherent light will become diffuse as they penetrate through multiple layers of cells or tissues. As a result, optical and photochemical islets may not have clear boundaries between treated and untreated volumes. Similarly, thermal islets may exhibit a temperature gradient from the center of the islet to its boundaries, and untreated tissue surrounding the islet also will exhibit a temperature gradient due to conduction of heat. Finally, damage islets can have irregular or indistinct boundaries due to partially damaged cells or structures or partially coagulated proteins. As used herein, therefore, the size of an islet within a lattice of islets, refers to the size of the islet as defined by the intended minimum or threshold amount of EMR energy delivered. As discussed in greater detail below, this amount is expressed as the minimum fluence, Fₘᵢₙ, and is determined by the nature of the cosmetic or medical application. For example, for photodynamic therapy, Fₘᵢₙ can be determined by the minimum fluence necessary to cause the desired photochemical reaction. Similarly, for increasing the permeability of the stratum corneum, Fₘᵢₙ can be determined by the minimum fluence necessary to achieve the desired SC temperature, and for destroying tissue, Fₘᵢₙ can be determined by the minimum fluence necessary to ablate the tissue or vaporize water. In each case, the size of the EMR-treated islet is defined by the size of the tissue volume receiving the desired minimum fluence.

Because of the scattering effects of tissue, the minimum size of an EMR-treated islet increases with the targeted depth in the tissue, ranging from several microns on the stratum corneum to several millimeters in subcutaneous tissue. For a depth of approximately 1 mm into a subject's tissue, the minimum diameter or width of an islet is estimated to be approximately 100 μm, although much larger islets (e.g., 1-10 mm) are possible. The size of a damage islet can be either smaller or larger than the size of the corresponding optical islet, but is generally larger as greater amounts of EMR energy are applied to the optical islet due to heat diffusion. For a minimum size islet at any particular depth in the skin, the wavelength, beam size, convergence, energy and pulse width have to be optimized.

C. The Depth of EMR-Treated Islets

The EMR-treated islets can be located at varying points within a tissue, including surface and subsurface locations, locations at relatively limited depths, and locations spanning substantial depths. The desired depth of the islets depends upon the intended cosmetic or medical application, including the location of the targeted molecules, cells, tissues or intercellular structures.

For example, optical islets can be induced at varying depths in a tissue or organ, depending upon the depth of penetration of the EMR energy, which depends in part upon the wavelength(s) and beam size. Thus, the islets can be shallow islets that penetrate surface layers of a tissue (e.g., 0-50 μm), deeper islets that span several layers of a tissue (e.g., 50-500 μm), or very deep, subsurface islets ((e.g., 500 μm-4 mm). Using optical energy, depths of up to 25 mm can be achieved using wavelengths of 1,000-1,300 nm. Using microwave and radio frequency EMR, depths of several centimeters can be achieved.

For thermal islets or damage islets, subsurface islets can be produced by targeting chromophores present at the desired depth(s), or by cooling upper layers of a tissue while delivering EMR. For creating deep thermal or damage islets, long pulse widths coupled with surface cooling can be particularly effective.

D. Fill Factor of EMR-Treated Lattices

In a given lattice of EMR-treated islets, the percentage of tissue volume which is EMR-treated is referred to as the “fill factor” or f, and can affect whether optical islets become thermal islets, damage islets or photochemical islets. The fill factor is defined by the volume of the islets with respect to a reference volume that contains all of the islets. The fill factor may be uniform for a periodic lattice of uniformly sized EMR-treated islets, or it may vary over the treatment area. Non-uniform fill factors can be created in situations including, but not limited to, the creation of thermal islets using topical application of EMR-absorbing particles in a lotion or suspension (see below). For such situations, an average fill factor (f_{avg}) can be calculated by dividing the volume of all EMR-treated islets Vᵢ^{islet} by the volume of all tissue Vᵢ^{tissue} in the treatment area,

$${f}_{avg} = {\sum }_{i} \frac{{V}_{i}^{islet}}{{V}_{i}^{tissue}} .$$

Generally, the fill factor can be decreased by increasing the center-to-center distance(s) of islets of fixed volume(s), and/or decreasing the volume(s) of islets of fixed center-to-center distance(s). Thus, the calculation of the fill factor will depend on volume of an EMR-treated islet as well as on the spacing between the islets. In a periodic lattice, where the centers of the nearest islets are separated by a distance d, the fill factor will depend on the ratio of the size of the islet to the spacing between the nearest islets d. For example, in a lattice of parallel cylindrical islets, the fill factor will be:

$$f = {π ( \frac{r}{d} )}^{2} ,$$

where d is the shortest distance between the centers of the nearest islets and r is the radius of a cylindrical EMR-treated islet. In a lattice of spherical islets, the fill factor will be the ratio of the volume of the spherical islet to the volume of the cube defined by the neighboring centers of the islets:

$$f = \frac{4 π}{3} {( \frac{r}{d} )}^{3} ,$$

where d is the shortest distance between the centers of the nearest islets and r is the radius of a spherical EMR-treated islet. Similar formulas can be obtained to calculate fill factors of lattices of islets of different shapes, such as lines, disks, ellipsoids, rectanguloids, or other shapes.

Because untreated tissue volumes act as a thermal sink, these volumes can absorb energy from treated volumes without themselves becoming thermal or damage islets. Thus, a relatively low fill factor can allow for the delivery of high fluence energy to some volumes while preventing the development of bulk tissue damage. Finally, because the untreated tissue volumes act as a thermal sink, as the fill factor decreases, the likelihood of optical islets reaching critical temperatures to produce thermal islets or damage islets also decreases (even if the EMR power density and total exposure remain constant for the islet areas).

The center-to-center spacing of islets is determined by a number of factors, including the size of the islets and the treatment being performed. Generally, it is desired that the spacing between adjacent islets be sufficient to protect the tissues and facilitate the healing of any damage thereto, while still permitting the desired therapeutic effect to be achieved. In general, the fill factor can vary in the range of 0.1-90%, with ranges of 0.1-1%, 1-10%, 10-30% and 30-50% for different applications. The interaction between the fill factor and the thermal relaxation time of a lattice of EMR-treated islets is discussed in detail below. In the case of lattices of thermal islets, it can be important that the fill factor be sufficiently low to prevent excessive heating and damage to islets, whereas with damage islets it can be important that the fill factor be sufficiently low to ensure that there is undamaged tissue around each of the damage islets sufficient to prevent bulk tissue damage and to permit the damaged volumes to heal.

### III. Devices For Creating Photoselective Islets

A. Exemplary Devices

In one embodiment, referring to FIGS. 13 and 14, a device 1050, capable of creating lattices of photoselective islets, passes electromagnetic radiation 1052 from a radiation source 1054, along an optical assembly and out an aperture 1058 at an end of optical assembly. Device 1050 functions as a laser handpiece that is designed to be attached to a base unit via an umbilical chord that supplies EMR, power and cooling to the handpiece. However, many other embodiments are possible, such as devices in which essentially all of the components are included in the handpiece and in which no base unit is required. Such alternate embodiments may be particularly suited as consumer devices for use in the home or other locations by non-professionals.

Electromagnetic radiation 1052 can be any radiation useful for selectively treating tissue, including EMR having wavelengths from approximately 200 nm to 10 mm (although, in some embodiments, wavelengths outside this range can be used). Additionally, more than one wavelength, ranges of wavelengths and/or bands of wavelengths can be employed.

Similarly, EMR can be generated using one or more coherent and/or non-coherent sources of EMR, such as EMR generated from lasers (including diode lasers and fiber lasers) and from lamps (such as flash lamps and halogen lamps). The lattices can also be produced using other types of sources. For example, microwave, radio frequency and low frequency or DC EMR sources can be used as energy sources to create lattices of EMR-treated islets. In addition, for treating tissue surfaces, the tissue surface can be directly contacted with heating elements in the pattern of the desired lattice.

In still other embodiments, various techniques can be used to obtain the desired wavelength or wavelengths, including filtering, frequency shifting, frequency doubling, etc. In some alternate embodiments, other forms of energy can be used, both alone and in combination (including in combination with EMR), for example, acoustic energy and ultrasound.

Though a broad range of wavelengths can be used in various embodiments, EMR having wavelengths in the range of 290-1800 nm (or even more preferably 530-1300 nm) are believed to be most suitable for creating photoselective islets in skin and other tissue of the human body. (Wavelengths lower than 290 nm are possible but are not recommended, because, although such lower wavelengths are suitable for use with various embodiments, the lower wavelengths are considered potentially carcinogenic and may have an adverse affect on the tissue in that regard.) In device 1050, radiation source 1054 of device 1050 is a Nd:YAG solid state laser that generates EMR having a wavelength of 1064 nm. Electromagnetic radiation 1052 is generated in the base unit, transmitted through a fiber 1060, emitted from radiation source 1054 (which is a fiber tip at the terminal end of fiber 1060).

Once emitted from radiation source 1054, electromagnetic radiation 1052 travels through an optical assembly. The optical assembly includes a collimating lens 1062, a first transmission tube 1064, an imaging lens 1066, a second transmission tube 1072, a lens array 1074, and aperture 1058 that serves as an opening through which the micro beams of EMR are transmitted. Aperture 1058 also includes an element 1080, which is a sapphire window located within the aperture 1058. The transmission tubes 1064 and 1072 are hollow cylindrical spaces containing air. Alternatively, the transmission tubes could include a waveguide and/or other optical elements.

In operation, electromagnetic radiation is emitted from radiation source 1054 and travels along the optical path. Lens 1062 collimates the electromagnetic radiation 1052, and lens 1066 subsequently images the electromagnetic radiation 1052. The electromagnetic radiation 1052 is uniform across the entire beam of radiation when it reached lens array 1074. Lens array 1074 transforms EMR 1052 from a single beam into an array of smaller beams 1078 that are directed through the optical window 1080 to the surface of the area of tissue being treated. Lens array 1074 is an array of micro-lenses having a numerical aperture (radius/focal depth) of 0.2-0.5. A suitable lens array is manufactured by SUSS MicroOptics SA. Lens array 1074 produces 75 beams of EMR each having a pitch of 1.3 mm (or alternatively 1.0 mm) and arranged in an orthogonal pattern.

Alternatively, the beams can be arranged in a hexagonal pattern, which would produce a total of 90 beams. Similarly, many other configurations of the beam array are possible. For example, in addition to hexagonal and orthogonal configurations, rectangular, circular, triangular, and other configurations could also be used. The various patterns have different advantages. For example, a hexagonal pattern would be preferable for providing greater beam densities, while an orthogonal pattern allows comparatively greater regions of untreated tissue between the volumes of treated tissue and/or allows relatively larger beam diameters.

Additionally, the pattern need not be uniform. Patterns can also be created by beams having varying relative cross-sectional areas and shapes can be used alone or in combination. For example, referring to FIGS. 15-17, an array of islets 1090 having a cross-sectional area roughly in the shape of an “X” could be used in various embodiments. One advantage of the “X” shaped islets is that the surface area of the islet is increased as opposed to islets with a circular cross section, which may be important in embodiments in which a healing or other response is desired because the ratio of surface area exposed to healthy tissue to damaged tissue is increased. Additionally, the density of the islets 1090 in an area of skin tissue can be controlled, for example, by overlapping the arms of the islets 1090. Similarly, oblong islets 192 and 194 can be formed. Like islets 1090, these islets provide increased surface area and the ability of control the density of the islets by, e.g., lining up the islets 192 in straight rows or staggering the islets 194 by lining them up in rows in which the oblong islets 194 are set at an angle relative to the direction of the rows and columns. Oblong beam shapes and islets may also irradiate the target more efficiently by increasing the probability of irradiating a chromophore within the tissue to create a photoselective islet (in comparison to beam shapes and islets that are circular).

Referring to FIG. 18, islets 1096 are cross-sectional ring structures. Islets 1096 can form rings or cylinders in the tissue, depending on the axial length of the ring/cylinder. The ring structures increase the surface area of the damaged tissue by providing an inner surface and outer surface that are both exposed to untreated tissue. The ring structures 1096 can be created several ways. For example, a circular lens can be used, or a device can include a predefined spot size spaced a distance from centerpoint and irradiate the tissue while rotating the spot about the centerpoint (e.g., by scanning or by a rotatable mirror). Referring to FIG. 19, in an alternative embodiment, a combination of rings 1096 and circular cross sections 198 can be used, as can other combinations of islet structures. Many other embodiments, both regularly and irregularly patterned, are possible.

As discussed above in conjunction with FIGS. 13-14, aperture 1058 includes an optical element 1080, which is a sapphire window in the present embodiment. The sapphire window 1080 is also part of a cooling system of device 1050. The window is attached to two cooling tubes 1084, which, during operation, supply and remove coolant respectively. (Note that only one cooling tube 1084 is shown in the cross-sectional view of device 1050, with the other tube 1084 located in the other half of the device that is not pictured in FIG. 14.) Thus, the window is in thermal communication with a chiller (not shown) that is contained in the base unit. The cooling system allows the sapphire to perform several functions. When levels of cooling are moderate, the sapphire window 1080 provides a cooling sensation that can reduce the level of pain that is perceived by the subject when the tissue is treated. Additionally, when more intense cooling is used, the sapphire window 1080 can be used to cool the tissue to a selected depth, depending on the intensity and duration of the cooling. The cooling system also serves to cool the device 1050.

Although not required, it is preferable during operation to flatten, compress or stretch the tissue along aperture 1076, to allow the device 1050 to uniformly irradiate the area of tissue with the array of micro-beams. Thus, the use of such techniques improves the precision of the device and allows it to create smaller islets holes and/or consistently sized, spaced and shaped islets. For example, referring to FIGS. 4 (discussed above) and 20, even a relatively flat area of skin will have significant variations in surface terrain 926 that can affect the alignment of the tissue surface relative to the focal plane W of one or more micro beams relative to the skin surface. The fluctuations in the surface terrain of the tissue will begin to be a greater percentage of the focal depth for smaller islets. Thus, the variation in skin surface terrain will have a greater impact as the size of the diameter of the islets decreases. Although it is not essential that the skin surface be aligned within the range of the focal depth Z0, it may be preferable in some embodiments to align the skin within that range to more precisely control the formation of, e.g., micro-holes or small damage islets.

Referring to FIG. 21, by using an even surface such as a sapphire window 932 (which can be flat or contoured), the surface of the tissue 928 can be precisely aligned with the focal plane of the beams to allow uniform islets to be created. Although, this precision is advantageous when forming small islets (such as micro-holes), precisely aligning the tissue may be beneficial in some embodiments that produce relatively larger islets. While such an alignment may produce superior results when forming small micro-holes and other small islets, such a device or structure is not required. In an alternative embodiment, the cooling surface, such as a window, grating or cooling plate, includes holes aligned with the array of beams, such that the untreated surface of the tissue is cooled without cooling the surface of the tissue that is irradiated by the array of beams. Such an embodiment would allow the tissue to be cooled to reduce any discomfort resulting from the treatment without cooling the surface of the irradiated tissue.

Referring again to FIGS. 13 and 14, in operation, the surface of the tissue to be treated is pressed against aperture window 1080, and the array of micro beams irradiate the surface. In the present embodiment, a safety mechanism (in this case a contact sensor) is included to prevent the laser from firing when the tissue is not in contact with the window 1080. That will prevent, among other things, the condition where the laser is accidentally fired while the window 1080 is off the surface of the tissue.

Many other configurations are possible. For example, an alternative optical assembly could result in the micro beams exiting the device in a parallel or a slightly divergent orientation, to prevent the array of micro beams from being applied to the surface at a greater intensity, thereby potentially damaging the tissue to an excessive degree) due to the convergence of the micro beams at the exit the device. Similarly, a diffusing device could be used.

During operation, lens array 1074 focuses the radiation having a wavelength of approximately 1064 nm in an orthogonal pattern within a generally circular treatment region (or footprint on the treated tissue) that measures 10 mm in diameter. The resulting treatment area is approximately 0.75 cm2. Each beam produced by the micro lens array has a spot size of 100-500 μm in diameter at the surface of the tissue. The spacing between each spot at the surface of the tissue is approximately 1000 μm. The EMR is applied at a fluence of 30 to 300 mJ per beam, and the variation in energy from spot to spot is designed to not exceed 20% but is generally less. The pulse duration is 3 to 20 ms, and the repetition rate is 0.2 to 0.5 Hz. The average laser pumping power is up to approximately 300 W at an energy of up to 1000 J. The depth of penetration of the EMR is approximately, 200-1000 μm. The cooling temperature is approximately 17° C., which provides sufficient cooling to prevent patient discomfort. Typically, a suitable optical impedance matching lotion or other suitable substance would be applied between aperture 1058 and the tissue being treated to provide enhanced optical and thermal coupling, but such a lotion is not required.

The specifications provided above are exemplary only, and many various and other combinations are possible, including greater or lesser numbers of beams, spot size, output energy per beam, wavelengths, depth of penetration, etc. Although many other ranges are possible, the ranges of specifications that are thought to be most suitable are: wavelengths in the range of 290-1300 nm (with the optimal wavelength(s) depending on the application); energy per beam of 1 nJ (nanojoule)-10 J (or more preferably 0.5 J); beam diameter of approximately 50 μm to 3 mm (or more preferably 250 μm); pulsewidths of one femtosecond to 1 second (or more preferably 10-60 ms); fill factors of 0.1% to 50%; beam size of 1 μm to 1 mm; pitch of 0.5 to 3.0 mm. The optimal specifications for a particular embodiment will vary depending on the application.

Furthermore, in still other embodiments, the EMR from the energy source can be focused by an optical device and/or shaped by masks, filters, optics, or other elements in order to create islets of treatment on the subject's skin. Other embodiments could include different combinations, types and number of optical components. Other embodiments could be configured to irradiate the tissue without the device being in contact with the tissue or by having an offset or spacer that spaces a transmission opening or other source of radiation some distance from the surface of the tissue during operation. In yet another embodiment, there is no cooling mechanism such that there is only passive cooling between the contact plate and the skin, or there may be no cooling at all.

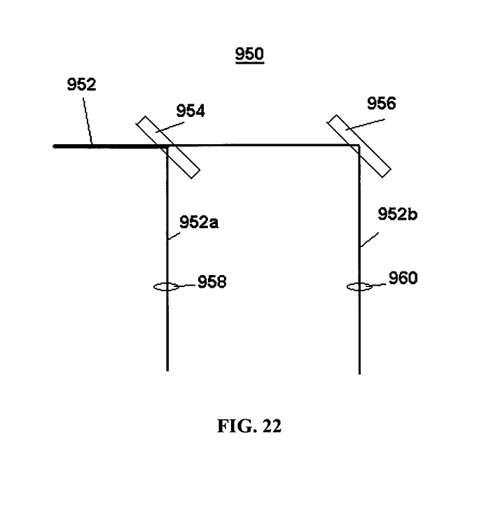

Additionally, other embodiments could include mechanisms other than lens arrays, such as scanning devices, partially reflective mirrors, etc. For example, one alternate embodiment could include a scanner that uses a single beam or several beams repeatedly to create the columns of damage in the tissue. Similarly, referring to FIG. 22, an array of mirrors 950 could be used. In this particular embodiment, a beam of EMR 952 passes through a set of mirrors that create a set of sub-beams 952a and 952b. EMR 952 passes through a first mirror 954 oriented at an angle of 45 degrees relative to the path of the EMR 952. Mirror 954 reflects approximately 50 percent of EMR 952 at a ninety degree angle to form sub-beam 952a and allows nearly all of the remaining portion of EMR 952 to pass through mirror 954 to create sub-beam 952b. Sub-beam 952b travels to a second mirror 956 that is nearly 100 percent reflective. Second mirror 956 also is oriented at an angle of 45 degrees relative to the path of the EMR 952 and reflects sub-beam 952b at a ninety degree angle and parallel to sub-beam 952a. Both beams travel through lenses 958 and 960 respectively. Lenses 958 and 960 focus the sub-beams 952a and 952b onto the tissue. Although the present embodiment creates two sub-beams, many different configurations and combinations of configurations are possible.

Furthermore, the characteristics of the resulting columns can be controlled by modulating the pulses of the micro-beams that are applied to the tissue. This can be done, for example, spatially or temporally. In some embodiments, the spatial geometry of the micro-beams can be designed to create resulting columns having specific characteristics. In other words, by varying the geometry of the micro-beams, including the overall pattern, the shape of the individual micro-beams and/or the combination of differently shaped micro-beams, the dimensions and other characteristics of the resulting columns of damage in the treated tissue can be controlled. For example, by increasing the relative cross-sectional area of the individual micro-beams, the depth of the columns into the tissue can be increased.

As another example, the shape of the footprint that the EMR islets form on the tissue can be varied to suit a particular application. For example, the footprint of the array of beams in device 1050 is roughly circular. There are various methods to control the shape of the footprint. In a scanning system, the system can be programmed to direct the beams in a pre-designated pattern. Similarly, in embodiments using an optical system similar to that of device 1050, the beam of EMR can be conditioned prior to passing through the lens array to have the desired cross-sectional shape.

In alternate embodiments, devices can use various sensors and feedback mechanisms to control and/or optimize the treatment. For example, speed sensors, contact sensors, imaging arrays, and controllers to aid in various functions of applying EMR to the patient's skin can be included. An optical detector, such as a capacitive imaging array, a CCD camera, a photodetector, or other suitable detector for a selected characteristic of the patient's skin can also be included. The output from such a detector can be applied to a controller, which is typically a suitably programmed microprocessor or other such circuitry, but may be special purpose hardware or a hybrid of hardware and software. The controller can, for example, control the turning on and turning off of an EMR source or other mechanism for exposing the tissue (e.g., skin) to the EMR. The controller may also control the power profile of the radiation. A controller can also be used, for example, to control the focus depth for the optical system and to control the portion or portions to which radiation is focused/concentrated at any given time. Similarly, a controller can be used to control the cooling element to control both the skin temperature and the cooling duration, both for pre-cooling and during irradiation.

One alternate embodiment is a consumer handheld device for performing fractional photothermolysis by treating, for example, pigmented lesions, vascular lesions and/or acne. The device includes a laser diode bar that is pulsed as the aperture of the device is moved or slides across the surface of the tissue. Such a device would preferably produce wavelengths in the range of 290-1300 nm (e.g., 800 nm), although other wavelengths are possible. The energy per micro beam would preferably be 1 nJ-1 J, depending on the size and number of beams. The pulsewidth would preferably be one femtosecond to 1 second. The fill factor would preferably be in the range of 0.1% to 50%. The beam size would preferably be 1 micrometer to 1 millimeter. Alternatively, a consumer device could include an EMR source that is a stack of laser diode bars that irradiate a predetermined spot size with an array of micro beams, e.g., a spot size of 10 mm by 10 mm. Such a device could be used in stamping mode (i.e., the aperture of the device is placed over the target area, the device is fired, and the device is then moved to the next target area), or it could be used in sliding mode. In still another embodiment, a single diode laser could be provided with a scanning mechanism to create an array of islets by sequentially firing the diode laser at it is moved or scanned in a predetermined pattern. Additional relevant disclosure related to consumer devices and methods for using such devices can be found in U.S. patent application Ser. No. 11/682,645, entitled Photocosmetic Device, filed Mar. 6, 2007, and in U.S. Provisional Patent Application 60/857,154, entitled Methods and Products for Producing Lattices of EMR-Treated Islets in Tissues, and Uses Therefore, filed Nov. 6, 2006., both of which are incorporated herein by reference.

B. Operation and Simulation Of Devices For Creating Photoselective

### Islets

In operation, device 1050 emits EMR in an array of beams separated by a fixed distance. The beams are created by dividing a beam of EMR by passing it through an array of micro-optical refractive elements. The redistribution of the optical energy into the beams results in large volumes of tissue between the beams that is untreated, which significantly increases safety margin of a single treatment pass. The efficacy of treatment may be enhanced by administering EMR to the tissue in multiple passes. Additionally, by creating space between the beams of EMR, the EMR can be spread over a larger area, and, thus, the spot size of the treated area can be larger for the same amount of energy applied. This may provide for faster treatment of relatively large areas and volumes of tissue. This can be important for some applications, such as the treatment of port-wine stains, which typically occupy relatively large areas at the surface of skin tissue.

In the case of device 1050, the effective fluence over the treatment area is reduced in comparison to existing products available on the market, due to the increased treatment area. For reference, Table 1 shows a comparison of the specifications of device 1050 and an existing non-fractional device on the market: the Lux1064™ handpiece manufactured by Palomar Medical Technologies, Inc. The same optical power train is used in both handpieces, including the same type of pumping flash lamps, reflectors, laser source assemblies, and laser rods.

TABLE 1
Comparative technical specifications of Lux1064 and Device 1050

| | | Non-fractional | |
|---|---|---|---|
| Parameter | Unit | device | Device 1050 |
| Wavelength | μm | 1.06 | 1.06 |
| Pulse duration | ms | 0.5 to 300 | 0.5 to 300 |
| Maximal total output | J | 41 | 41 |
| energy | | | |
| Treatment spot diameter | mm | 1.5 to 6 | 10 |
| Maximal effective | J/cm² | 500 | 55 |
| fluence over treatment | | | |
| spot | | | |
| Maximal output energy | J | 41 | 1 |
| per beam | | | |
| Overall number of beams | — | 1 | 40 to 90 |
| Cooling temperature | Deg C. | 17 | 17 |
| Repetition rate | Hz | 0.2 to 1 | 0.2 to 1 |
| Maximal pumping | J | 1500 | 1500 |
| energy | | | |
| Average pumping power | W | 300 | 300 |
| Output energy | — | single-beam with | multi-beam with |
| distribution profile | | less than 20% | less than 20% |
| | | variation over | beam-to-beam |
| | | beam area | variation |
| Output distribution | — | Decaying | Decaying |
| temporal profile | | trapezoid | trapezoid |

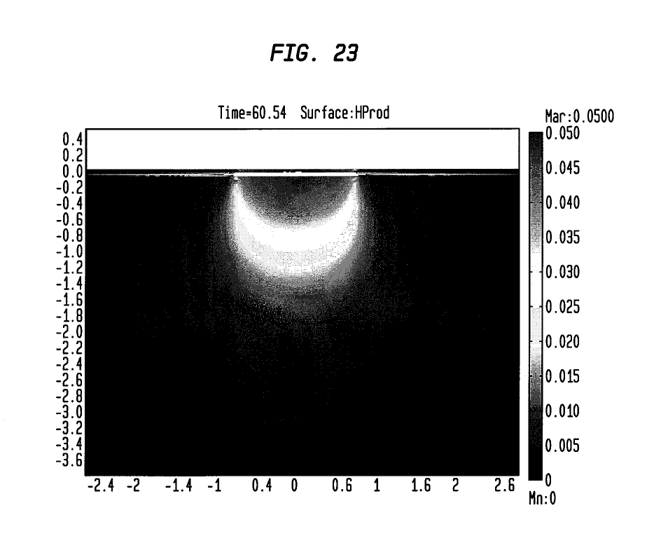

A series of computer simulations show the tissue effects of device 1050 when compared to a non-fractional device—again, the Luxi1064™ handpiece. The simulations were created using a proprietary opto-thermal computer model described in greater detail in G. Altshuler, M. Smimov and I. Yaroslavsky, “Lattice of optical islets: a novel treatment modality in photomedicine,” J. Phys. D: Appl. Phys. 38 No 15 (Aug. 7, 2005) 2732-2747, which incorporated herein by reference. The most reliable values of thermal and optical properties for tissues available from literature were used in the simulations. Arrhenius formalism was employed to compute tissue damage. A 1.5 mm diameter, 500 J/cm² regime was simulated for Lux1064, and 0.45 J/micro-beam regime (corresponding to highest density, 90 microbeams configuration) was simulated for device 1050. The results are illustrated in FIGS. 23 to 28. As can be seen, device 1050 does not generate a higher temperature in tissue than the non-fractional device. Further, the EMR-treated islets produced by device 1050 do not form a continuous, confluent damage area, thus in this case leaving lattices of undamaged tissue between the beams.

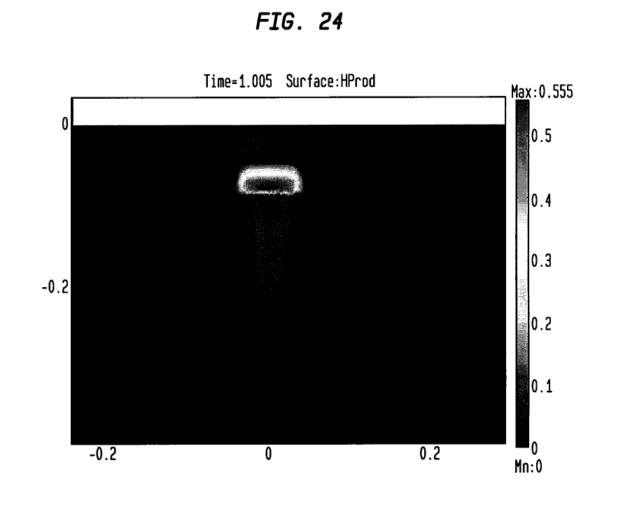

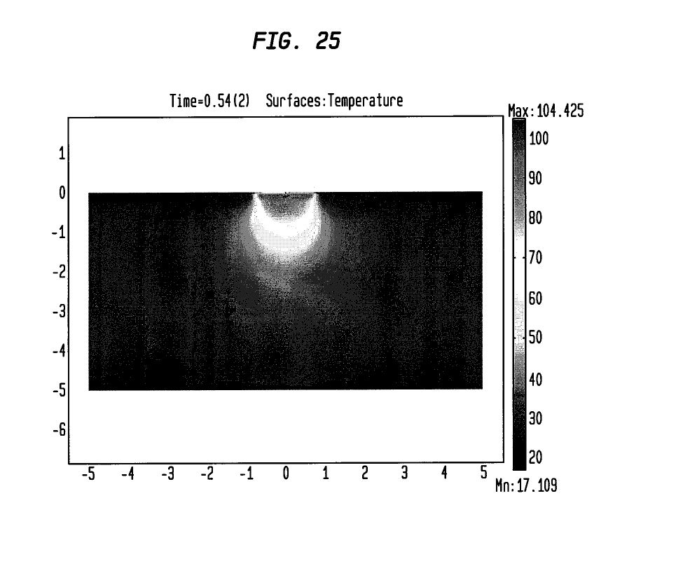

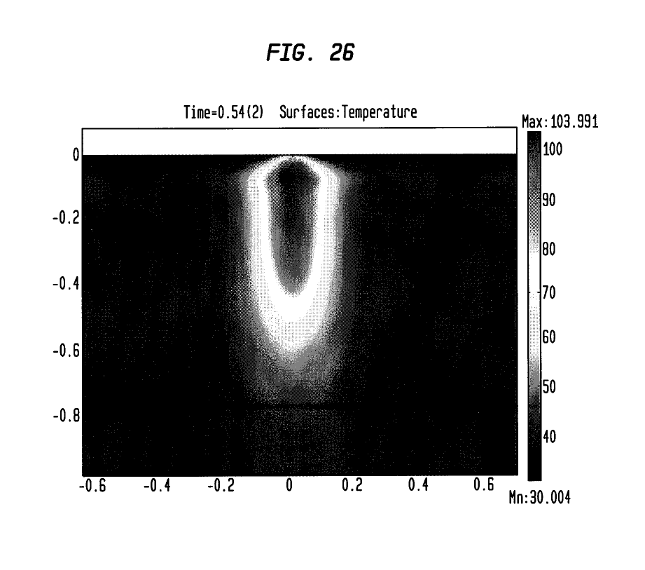

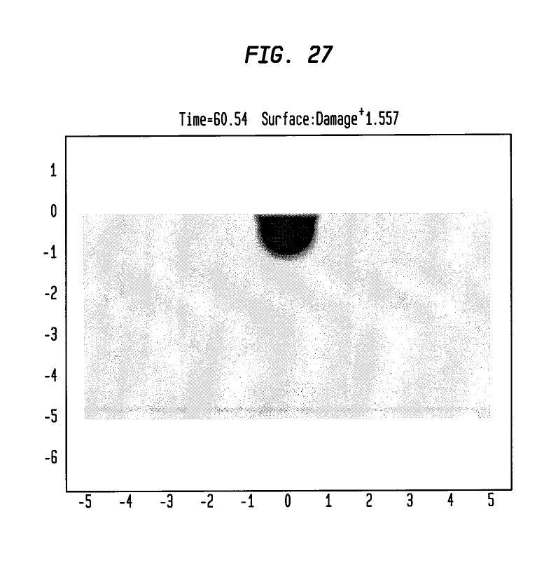

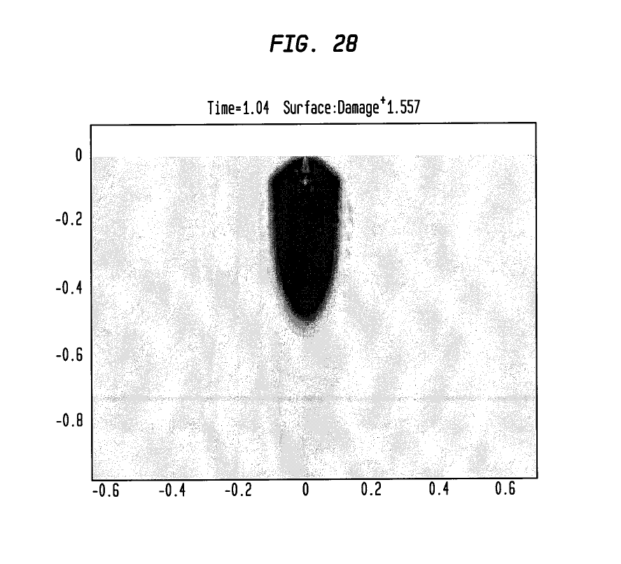

FIG. 23 shows that heat source distribution in tissue produced by the non-fractional device, and FIG. 24 shows the same distribution for a single micro beam of device 1050. The dimensions along both the horizontal and vertical axes are in millimeters, but the spatial scales of the figures are not identical. The temperature in both cases extends from approximately 100 degrees Celsius in the center (noted by the darker shades), to lower temperatures of approximately 50 degrees Celsius (noted by the lightest shades) to ambient body temperature (again noted by a darker background shade). FIG. 25 illustrates the temperature field distribution in tissue for the non-fractional device, and FIG. 26 illustrates the temperature field distribution in tissue for a single beam of device 1050. The dimensions along both the horizontal and vertical axes are in millimeters, but the spatial scales of the figures are not identical. The temperature in both cases extends from approximately 100 degrees Celsius in the center (noted by the darker shades), to lower temperatures of approximately 50 degrees Celsius (noted by the lightest shades) to ambient body temperature (again noted by a darker background shade). FIG. 27 illustrates the resulting tissue damage for a non-fractional device, and FIG. 28 illustrates the resulting tissue damage for a single micro beam of device 1050. The dimensions along both the horizontal and vertical axes are in millimeters, but the spatial scales of the figures are not identical.

Additional exemplary treatment parameters are shown in Table 2 below.

TABLE 2
Exemplary treatment parameters.

| | | | | |
|---|---|---|---|---|
| Damage | 1 | 2 | 3 | 5 |
| heating depth, | | | | |
| mm | | | | |
| Damage/heated | 0.2-3 | 0.5-5 | 0.75-6 | 1-10 |
| zone diameter, | | | | |
| mm | | | | |
| Wavelength, | 900-1850 | 900-1400 | 900-1350 | 900-1300 |
| nm | 2080-2300 | 1500-1750 | | |
| Beam diameter | 0.5-8 | 1-10 | 2-15 | 3-25 |
| (2D beam) or | | | | |
| width (1D | | | | |
| beam) , mm | | | | |
| Fill factor* | 0.01-0.5 | 0.01-0.3 | 0.01-0.3 | 0.01-0.3 |
| Pulse width, s | 0.001-10 | 0.1-20 | 0.5-30 | 1-120 |
| Precooling | 0-10 | 0-20 | 0-60 | 0-100 |
| time, s | | | | |
| Postcooling | 0-20 | 0-30 | 0-60 | 0-120 |
| time, s | | | | |
| Input power | 5-100 | 3-70 | 1-50 | 0.5-35 |
| density, W/cm² | | | | |

*Fₘₐₓ is the maximum possible fill factor, that is, the ratio of the light
exposed area to the total area of the treatment site,
$F = \frac{π}{4} \cdot {( \frac{D}{d} )}^{2} ,$
where D is the spot diameter, d is the spot separation.

### IV. Applications of Devices and Methods for Creating Photoselective Islets

Device 1050 and other embodiments can employ both wavelength and spatial selectivity to treat targets within the skin as well as other types of tissue. For example, by selecting a wavelength that selectively absorbed by hemoglobin, device 1050 can be used to treat, for example, port wine stains. The tissue volume containing the port wine stain lesion is treated. The hemoglobin in the tissue, which resides essentially in a pool below the surface of the tissue, will selectively absorb the EMR within the space that the pool occupies. The surrounding tissue, which does not absorb the EMR to the same degree, is unaffected or is affected to a much lesser degree. Thus, upon treatment, an array of photoselective islets is created within the vascular lesion, and not in the surrounding tissue. The damage islets act to cut the supply of blood to the lesion, which dries up and is removed by natural processes of the body or by further treatments to remove the treated tissue.

Similarly, other pigmented and vascular lesions can be treated. Other treatments include, but are not limited to, removal of tattoos, tumors, varicose veins and other blood vessels.

The creation of lattices of damage islets can be used to remove tattoos by killing the cells containing the tattoo ink particles (typically cells of the upper dermis). After these cells are killed, the tattoo ink is cleared away from the tissue site by normal scavenging processes. Alternatively, or in addition, lattices of damage islets can be used to remove tattoos by selecting the wavelength(s) of the EMR treatment to cause selective absorption of the EMR energy by the tattoo ink particles. In some embodiments, the pulse width of the incident pulse is chosen to match the thermal relaxation time of the ink particles. The absorption of the EMR energy by the tattoo ink particles can cause the cells to be heated and killed; can cause the ink particles to undergo photobleaching or be broken into smaller molecules which are removed by normal processes; or can otherwise cause the ink to be destroyed.

Furthermore photoselective islets can be used in a variety of applications in a variety of different organs and tissues. For example, EMR treatments can be applied to tissues including, but not limited to, skin, mucosal tissues (e.g., oral mucosa, gastrointestinal mucosa), ophthalmic tissues (e.g., conjuctiva, cornea, retina), and glandular tissues (e.g., lacrimal, prostate glands). As a general matter, the methods can be used to treat conditions including, but not limited to, lesions (e.g., sores, ulcers), acne, rosacea, undesired hair, undesired blood vessels, hyperplastic growths (e.g., tumors, polyps, benign prostatic hyperplasia), hypertrophic growths (e.g., benign prostatic hypertrophy), neovascularization (e.g., tumor-associated angiogenesis), arterial or venous malformations (e.g., hemangiomas, nevus flammeus), and undesired pigmentation (e.g., pigmented birthmarks, tattoos).

In some embodiments, thermal islets can be produced which span from a tissue surface to deeper layers of the tissue, or which are present entirely in subsurface layers. Such thermal islets can be used for applications such as thermally-enhanced photobiomodulation, photobiostimulation and photobiosuspension, as well as the creation of damage islets, as described below.

Some embodiments provide methods of treating tissues by creating lattices of damage islets. These methods can be used in, for example, skin rejuvenation, tattoo removal (e.g., killing cells containing ink particles, ablation of tattoo ink particles), acne treatment (e.g., damaging or destroying sebaceous glands, killing bacteria, reducing inflammation), pigmented lesion treatment, vascular lesion treatment, and nevus flammeus (“port wine stain”) removal (e.g., reducing pathological vasculature), among others. Lattices of damage islets can also be used to increase the permeability of the stratum corneum. The time for recovery or healing of such damage islets can be controlled by changing the size of the damage islets and the fill factor of the lattice.

In one embodiment, a method of tissue remodeling is based on controlled tissue damage. One embodiment of tissue remodeling is skin “rejuvenation,” a complex process involving one or more of (a) reduction in skin dyschromia (i.e., pigment non-uniformities), (b) reduction in telangiectasia (i.e., vascular malformations), (c) improvement in skin texture (e.g., pore size reduction and improvement in surface roughness), (d) reduction of rhytides and wrinkles, skin smoothing, and (e) improvement in skin tensile properties (e.g., increase in elasticity, lifting, tightening).

The creation of lattices of damage islets can result in skin lifting or tightening as a result of (a) shrinkage of collagen fibrils subjected to elevated temperatures (immediate effect) or (b) coagulation of localized areas in the dermis and hypodermis (immediate to short-term effect).

The creation of lattices of damage islets can result in smoother skin texture as a result of coagulation of localized areas in the dermis and hypodermis (immediate to short-term effect). This technique also can be used for texturing tissues or organs other than the dermis/epidermis (e.g., lip augmentation).

The creation of lattices of damage islets can result in the promotion of collagen production as a result of the healing response of tissues to thermal stress or thermal shock (medium- to long-term effect).

The creation of lattices of damage islets can be used to treat acne by selecting the wavelength(s) of the EMR treatment to cause selective absorption of the EMR energy by sebum, or targeting the lattice to sebaceous glands, in order to selectively damage or destroy the sebaceous glands. The EMR treatment can also be targeted to bacteria within acne sores.

The creation of lattices of damage islets can be used to treat hypertrophic scars by inducing shrinkage and tightening of the scar tissue, and replacement of abnormal connective tissue with normal connective tissue.

The creation of lattices of damage islets can be used to treat warts and calluses by selectively targeting the pathological tissue to kill cells or cause tissue peeling. The pathological tissue can be replaced with normal tissue by normal biological processes.

The creation of lattices of damage islets can be used to decrease the time needed for the healing of wounds or burns (including frostbite) by increasing the wound or burn margin without substantially increasing the volume.

The creation of lattices of damage islets can be used to reduce cellulite by changing the mechanical stress distribution at the dermis/hypodermis border. Alternatively, or in addition, lattices of damage islets can be used to reduce fat in the hypodermis (subcutaneous tissue) by heating and damaging fatty cells inside islets.

The creation of lattices of damage islets can be used in order to decrease the amount or presence of body hair by targeting lattices of damage islets to hair follicles in the skin. The methods can selectively target melanin or other chromophores present in hair or hair follicles, or may non-selectively target water in the hair follicle.

The creation of lattices of damage islets can be used in order to damage or destroy internal epithelia to treat conditions such a benign prostatic hyperplasia or hypertrophy, or restenosis. The methods can also be used to weld tissues together by creating damage areas at tissue interfaces.

### EQUIVALENTS

While only certain embodiments have been described, it will be understood by those skilled in the art that various changes in form and details may be made therein without departing from the spirit and scope as defined by the appended claims. Those skilled in the art will recognize, or be able to ascertain using no more than routine experimentation, many equivalents to the specific embodiments described herein. Such equivalents are intended to be encompassed in the scope of the appended claims.

### REFERENCES AND DEFINITIONS

The patent, scientific and medical publications referred to herein establish knowledge that was available to those of ordinary skill in the art. The entire disclosures of the issued U.S. patents, published and pending patent applications, and other references cited herein are hereby incorporated by reference.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent or later-developed techniques which would be apparent to one of skill in the art. In addition, in order to more clearly and concisely describe the claimed subject matter, the following definitions are provided for certain terms which are used in the specification and appended claims.

### Numerical Ranges

As used herein, the recitation of a numerical range for a variable is intended to convey that the embodiments may be practiced using any of the values within that range, including the bounds of the range. Thus, for a variable which is inherently discrete, the variable can be equal to any integer value within the numerical range, including the end-points of the range. Similarly, for a variable which is inherently continuous, the variable can be equal to any real value within the numerical range, including the end-points of the range. As an example, and without limitation, a variable which is described as having values between 0 and 2 can take the values 0, 1 or 2 if the variable is inherently discrete, and can take the values 0.0, 0.1, 0.01, 0.001, or any other real values ≧0 and ≦2 if the variable is inherently continuous. Finally, the variable can take multiple values in the range, including any sub-range of values within the cited range.

Or. As used herein, unless specifically indicated otherwise, the word “or” is used in the inclusive sense of “and/or” and not the exclusive sense of “either/or.”

As used herein, EMR includes the range of wavelengths approximately between 200 nm and 10 mm.

The prefix “photo” refers to photons generated by EMR, and is not limited to EMR having optical and/or visible wavelengths. Thus, for example, the term “photoselective islets” refers to islets created by the selective absorption of photons of EMR.

Optical radiation, i.e., EMR in the spectrum having wavelengths in the range between approximately 200 nm and 100 μm, is preferably employed in some of the embodiments described above, but, also as discussed above, many other wavelengths of energy can be used alone or in combination. The term “optical” generally refers to energy in that range. For example, as used herein, the term “optical path” is a path suitable for transmitting “optical radiation.”

However, although many of the embodiments described herein are described with reference to optical radiation and optical elements within devices that employ optical radiation, those embodiments are not intended to be limiting. Other embodiments may employ EMR outside the optical range or employ other forms of energy to form treatment islets. Such embodiments may include elements other than optical elements. For example, EMR outside the optical range can be employed, and other sources such as ultrasound, microwave, acoustic, photo-acoustic and other sources of energy may also be used to form treatment islets in some embodiments. Thus, although the embodiments described herein are described with regard to the use of EMR and optical energy to form the islets, other forms of energy to form the islets are within the scope of the invention and the claims.

We claim:

1. A method for treating a subvolume of tissue located below a surface of the tissue comprising:
irradiating the tissue with optical radiation that is more readily absorbed by the subvolume of tissue than by portions of the tissue surrounding the subvolume;
wherein the optical radiation creates a plurality of treatment zones within the subvolume of tissue separated by substantially untreated tissue within the subvolume; and
wherein the portions of tissue surrounding the subvolume of tissue are substantially untreated.

2. The method of claim 1, wherein the treatment zones are regularly spaced from each other.

3. The method of claim 1, wherein the treatment zones have a width of between approximately 1 and 1000 micrometers.

4. The method of claim 1, wherein the treatment zones have a width of between approximately 30 and 100 micrometers.

5. The method of claim 1, wherein the treatment zones are located within the dermis.

6. The method of claim 1, wherein the treatment zones are located within the epidermis.

7. The method of claim 1, wherein the tissue is skin tissue.

8. The method of claim 1, wherein the subvolume of tissue is a lesion.

9. The method of claim 1, wherein the subvolume of tissue is a vascular lesion.

10. The method of claim 1, wherein the subvolume of tissue is a pigmented lesion.

11. The method of claim 1, wherein the subvolume of tissue is a vein.

12. The method of claim 1, wherein the treatment zones have a fill factor in a cross-sectional plane extending through the treatment zones of between approximately 1 percent and 90 percent.

13. The method of claim 1, wherein the treatment zones have a fill factor in a cross-sectional plane extending through the treatment zones of between approximately 1 percent and 50 percent.

14. The method of claim 1, wherein the optical radiation has a wavelength of 1064 nanometers.

15. The method of claim 1, wherein the optical radiation is coherent.

16. The method of claim 1, wherein the optical radiation is incoherent.

17. The method of claim 1, wherein the optical radiation includes a broadband range of wavelengths.

18. The method of claim 1, wherein the treatment zone is a zone of coagulated tissue.

19. The method of claim 1, wherein the treatment zone is a zone of thermally injured tissue.

20. The method of claim 1, wherein the treatment zone is a zone of denatured tissue.

21. The method of claim 1, wherein the treatment zone is a zone of ablated tissue.

22. The method of claim 1, wherein the substantially untreated portions of tissue contains zones of thermally heated tissue resulting from the step of irradiation.

23. A method for treating a subsurface tissue comprising:
irradiating a surface of a tissue with electromagnetic radiation that is transmitted to a subsurface tissue via an intervening tissue located between the surface and the subsurface tissue, the electromagnetic radiation being more preferentially selected by the subsurface tissue than by the intervening tissue;
wherein the electromagnetic radiation creates a plurality of damage zones within the subsurface tissue separated by undamaged tissue within the subvolume; and
wherein the intervening tissue is undamaged.

24. The method of claim 23, wherein the damage zones are regularly spaced from each other.

25. The method of claim 23, wherein the damage zones have a width of between approximately 1 and 1000 micrometers.

26. The method of claim 23, wherein the damage zones have a width of between approximately 30 and 100 micrometers.

27. The method of claim 23, wherein the damage zones are located within the dermis.

28. The method of claim 23, wherein the damage zones are located within the epidermis.

29. The method of claim 23, wherein the intervening tissue is skin tissue.

30. The method of claim 23, wherein the subsurface tissue is a lesion.

31. The method of claim 23, wherein the subsurface tissue is a vascular lesion.

32. The method of claim 23, wherein the subsurface tissue is a pigmented lesion.

33. The method of claim 23, wherein the subsurface tissue is a vein.

34. The method of claim 23, wherein the damage zones have a fill factor in a cross-sectional plane extending through the treatment zones of between approximately 1 percent and 90 percent.

35. The method of claim 23, wherein the damage zones have a fill factor in a cross-sectional plane extending through the treatment zones of between approximately 1 percent and 50 percent.

36. The method of claim 23, wherein the electromagnetic radiation has a wavelength of 1064 nanometers.

37. The method of claim 23, wherein the damaged zones are zones of coagulated tissue.

38. The method of claim 23, wherein the damaged zones are zones of thermally injured tissue.

39. The method of claim 23, wherein the damaged zones are zones of denatured tissue.

40. The method of claim 23, wherein the damaged zones are zones of ablated tissue.

41. The method of claim 23, wherein the step of irradiating the surface creates zones of thermally heated tissue within the undamaged intervening tissue.

42. The method of claim 1, wherein the step of irradiating further comprises irradiating the tissue by scanning the electromagnetic radiation to an array of locations on the surface of the tissue corresponding to the damaged zones within the subsurface tissue.

43. The method of claim 1, wherein the step of irradiating further comprises irradiating the tissue with an array of beam of electromagnetic radiation that create the damaged zones within the subsurface tissue.

44. A method for treating a subvolume of tissue located below a surface of the tissue comprising:
irradiating the tissue with electromagnetic radiation; and
creating an array of treatment zones within the subvolume of tissue separated by other tissue of the subvolume;
wherein a portion of tissue surrounding the subvolume includes zones of differently-treated tissue.

45. The method of claim 44, wherein the other tissue of the subvolume is untreated.

46. The method of claim 44, wherein the other tissue of the subvolume is heated.

47. The method of claim 44, wherein portions of the other tissue of the subvolume is heated by heat diffused from the treatment zones.

48. The method of claim 44, wherein the portion of tissue surrounding the subvolume is untreated.

49. The method of claim 44, wherein the portion of tissue surrounding the subvolume is heated.

50. The method of claim 44, wherein the portion of tissue surrounding the subvolume contains zones of heated tissue corresponding to the treatment zones of the subvolume.

51. The method of claim 44, wherein the treatment zones contain damaged tissue.

52. The method of claim 51, wherein the portion of tissue surrounding the subvolume contains zones of damaged tissue corresponding to the treatment zones of the subvolume, wherein the degree of damage in zone of damaged tissue in the portions of tissue surrounding the subvolume is less than the degree of damage in the treatment zones of the subvolume.

53. The method of claim 44, wherein the treatment zones contain coagulated tissue.

54. The method of claim 44, wherein the treatment zones contain denatured tissue.

55. A device for treating soft tissue comprising:
a source of electromagnetic radiation;
an output aperture;
a transmission path extending from the source of the electromagnetic radiation to the output aperture, and configured to deliver the electromagnetic radiation to the soft tissue;
wherein the output aperture is configured to emit electromagnetic radiation in a pattern of spots on a tissue surface; and
wherein the source is configured to generate electromagnetic radiation that is selectively absorbed by a subvolume of tissue located below a surface of the soft tissue.

56. The device of claim 55, wherein the source is configured to produce coherent radiation.

57. The device of claim 55, wherein the source is configured to produce radiation having a wavelength of between approximately 190 nanometers and 100 micrometers.

58. The device of claim 55, wherein the source is a laser.

59. The device of claim 55, wherein the source produces electromagnetic radiation having an infrared wavelength.

60. The device of claim 55, wherein the source produces electromagnetic radiation having a wavelength of approximately 1064 nanometers.

61. The device of claim 55, wherein the transmission path includes and array of lenses to simultaneously generate an array of beams of electromagnetic radiation corresponding to the pattern of spots on the tissue surface.

62. The device of claim 55, wherein the transmission path includes a scanning device to generate an array of beams of electromagnetic radiation corresponding to the pattern of spots on the tissue surface.
